(19) United States
(12) Patent Application Publication
(10) Pub. No.: US 2026/0159830 A1
(43) Pub. Date: Jun. 11, 2026

(54) Solid Support Comprising Ligands Suitable for Polynucleic Acid Processing, Articles and Methods

(71) Applicant: Solventum Intellectual Properties Company, Maplewood, MN (US)

(72) Inventors: Annabelle Watts, Minneapolis, MN (US); Tonya D. Bonilla, Lake Elmo, MN (US); Jerald K. Rasmussen, Woodville, MN (US); Joshua M. Fishman, Saint Louis Park, MN (US); Lindsay L. Traeger, Saint Paul, MN (US); Miguel A. Guerra, Woodbury, MN (US)

(21) Appl. No.: 18/707,692

(22) PCT Filed: Nov. 4, 2022
(86) PCT No.: PCT/IB2022/060641

§ 371 Date: May 6, 2024

Related U.S. Application Data
(60) Provisional application No. 63/278,536, filed on Nov. 12, 2021.

(51) Int. Cl. C12N 15/10

(52) U.S. Cl. CPC C12N 15/1093

(57) ABSTRACT

A solid support comprising ligands bound to the solid support; wherein at least a portion of the ligands comprises an amide group and a fluorinated amine group or a heterocyclic aliphatic group. The fluorinated amine group or a heterocyclic aliphatic group is typically an ionizable group. The amide group may be the reaction product of an acidic group or a salt thereof on the surface of the solid support and an amine compound, such as morpholine, piperazine, and derivatives thereof. As described is a kit comprising the solid support and a buffer having a ph of less than 5.5 and method of processing polynucleic acids.

### SUMMARY

In one embodiment, a solid support is described comprising ligands bound to the solid support. At least a portion of the ligands comprises a fluorinated or a heterocyclic aliphatic group. In typical embodiments, the ligand further comprises an amide group. In some embodiments, the solid support comprises particles such as magnetic particles or non-magnetic particles.

In some embodiments, the ligands bound to the solid support comprise an ionizable group, such as an amine group. The solid support comprising the ligands may be characterized by zeta potential and/or other polynucleic acid (e.g. DNA) processing test results. The amide group is typically the reaction product of acidic groups or a salt thereof on the surface of the solid support and an amine compound. In some embodiments the amine compound is morpholine, piperazine, and derivatives thereof. Thus the ligand compound is a reaction product of such amine compounds. In some embodiments, the amine compound comprises a secondary amine group. In some embodiments, the amine compound comprises a tertiary amine group.

In another embodiment a solid support is described comprising a ligand that is the reaction product of an amine compound wherein the amine compound comprises a fluorinated group or a piperazine derivative, as described herein.

In another embodiment, a kit is described comprising the solid support comprising ligands, as described herein, and a low pH buffer (e.g. having a pH of less than 5.5) and optionally other buffers such as a higher pH buffer (e.g. having a pH of greater than 6).

The solids supports and kits are suitable for use for methods of processing polynucleic acids. In one embodiment, the method comprises:
a) providing a solid support comprising ligands with an amide group as described herein;
b) exposing the solid support to polynucleic acid molecules in a low pH buffer (e.g. having a pH less than 5.5) to bind at least a portion of the polynucleic acid molecules to the ligands;
c) exposing the solid support with bound polynucleic acid molecules to a buffer having a higher pH (e.g. greater than 6) to release a portion of the bound polynucleic acid molecules from the ligands of the solid support and optionally retaining a portion of the polynucleic acid molecules bound to the solid support
d) optionally washing the solid support comprising the bound and/or retained polynucleic acids molecules (e.g. after step b) and/or after step c);
e) optionally preparing a suspension from the solid support comprising the retained bound polynucleic acids molecules;
f) utilizing the released portion of bound polynucleic acid molecules of c), and/or the retained portion of the polynucleic acid molecules bound to the solid support or the suspension thereof.

Utilizing the released and/or retained and/or suspended polynucleic acid molecules may comprise size separation, purification, quantification, detection or modification (e.g. via exposure to at least one enzyme) such as amplification, transcription, tagmentation, digestion, ligation, or preparation of libraries (e.g. for nucleic acid sequencing).

### DETAILED DESCRIPTION

The methods and articles described herein comprise a solid support. The surface of the solid support comprises ligands. The ligands can reversibly bond or otherwise interact with the polynucleic acids, including for example ionic/electrostatic interactions, hydrogen-bonding interactions, hydrophobic interactions, and combinations thereof.

Various solid supports have been described in the literature including particles (e.g. beads). Other solid support are known in the art.

The solid support is typically comprised of organic polymers (e.g. plastics) or inorganic materials or combinations of both. Examples of suitable solid support materials include metal oxides such as Al₂O₃, TiO₂, ZrO₂, Ta₂O₃; as well as silica materials such as SiO₂ and polysilicic acid. The solid supports can be magnetic materials such as iron, cobalt or nickel and oxides, alloys, ceramics or amalgams thereof. Suitable organic polymers include polystyrene, poly(meth)acryl polymers including poly(meth)acrylates and poly(meth)acrylamides, polyurethanes, polyamides such as nylon; polyolefins, such as polyethylene, polypropylene, polybutadiene, and copolymers thereof. Other solid support materials include polysaccharides, and in particular hydrogels such as agarose, cellulose, dextran, SEPHADEX®, SEPHACRYL®, and chitosan. Inorganic supports include, for example, glass or metal surfaces such as gold. In some embodiments, the ligands described herein (e.g. covalently) bond to the solid support material.

In some embodiments, the solid support is a plurality of particles including magnetic beads and in particular paramagnetic beads. In other embodiments, the solid support is typically non-magnetic particles, such as silica.

Various magnetic and non-magnetic particles can be utilized as a solid support. In some embodiments, the particles have a mean particle size of at least 0.5 or 1 micron. In some embodiments, the particles typically have a mean particle size no greater than 500, 250, 100, 75, 50, 25, 15, 10, or 5 microns. Although the particles are typically spherical, other shaped particles can also be utilized.

In typical embodiments, the (e.g. ionizable) ligand is formed by (e.g. covalently) bonding amine compounds, as described herein, to (para) magnetic particles (also referred to as beads) comprising carboxylic acid groups or salts thereof (e.g. carboxylate groups) on the surface of the particles. However, it is appreciated that such groups can be provided on other solid supports, as described above, for reaction with the amine compounds. The amide group could also be the reaction product of an amine and an ester. In yet other embodiments, a ligand with an amide group could be the reaction product of an amide and an acid chloride.

In some embodiments, solid supports comprising carboxylic acid groups or salts thereof (e.g. carboxylate groups) on the surface are commercially available. Some commercially available beads are described in the forthcoming examples. Gold nanoparticles (10 nm carboxylic acid functionalized polyethylene glycol 3000 g/mole) are commercially available from MilliporeSigma (Product No. 765457). Other commercially available products are carboxyl multiwell plates (Corning® PureCoat™ Carboxyl plate), carboxyl modified polystyrene (Polybead® Carboxylate Microspheres), carboxy-terminated biosensor surface (Octet® Amine Reactive 2nd-Generation Biosensors), carboxylic acid silica gels (SiliaBond Carboxylic Acid (WCX), product number R70030B), and carboxylate polystyrene monodisperse microspheres, commercially available from Polysciences as “Polybead® Carboxylate Sampler Kit.

As described for other amine compounds in US2019/0071662, the carboxylic acid groups or salts thereof on the surface of the solid support (e.g. particles) covalently bond with an amine group of an amine compound forming an amide linking group.

In some embodiments, carboxylic acid groups or salts thereof on the surface of the solid support (e.g. particles) are reacted with an amine compound comprising a heterocyclic aliphatic group. Representative amine compounds typically comprise 6-membered rings. Representative amine compounds include for example morpholine, piperazine, and derivatives thereof.

A representative particle, as an illustrative solid support, comprising a ligand is depicted as follows:

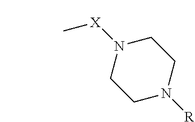

wherein X—N is an amide group;
R is hydrogen or an organic group.
   R typically comprises 1 to 20 carbon atoms. Representative organic groups include alkyl, substituted alkyl, aryl, substituted aryl, and combinations thereof. The organic group may be linear, branched, and may optionally comprise an aliphatic or aromatic cyclic group. Representative substituents include hydroxy, alkoxy, halo, ether, thioether, phenyl, benzyl, pyridinyl, nitro, cyano, sulfonyl, ester and combinations thereof. In some embodiments, the organic group comprises a fluorinated group such as a fluorinated alkyl group. In some embodiments, the (optionally fluorinated) organic group comprises no greater than 8, 6, 4, or 3 carbon atoms.

In typical embodiments, X of X—N is —C(O)—, resulting in —C(O)N—, an amide linking group prepared by the reaction of a carboxylic acid or carboxylate group with an amine.

A representative reaction scheme is depicted as follows:

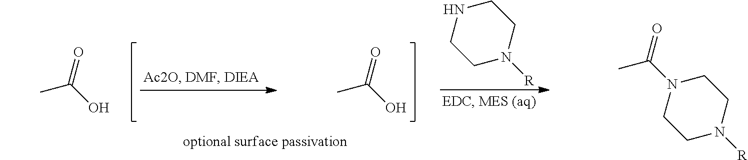

In some embodiments, the surface of the solid support (e.g. particles) is subject to passivation prior to reaction with the amine compounds. Passivation involves reacting surface functionality present on the solid support capable of positive ionization in aqueous buffer with a chemistry that prevents such ionization. In some embodiments, the solid supports are passivated with acetic anhydride.

Some representative (e.g. particle) solid supports prepared from piperazine, N-methyl piperazine, and N-phenyl piperazine are depicted as follows:

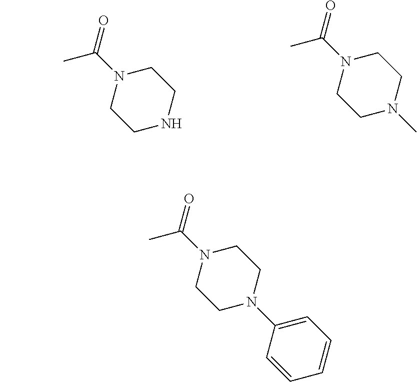

Other suitable heterocyclic amine compounds include, for example, N-(2-hydroxyethyl)piperazine, N-(4-methoxyphenyl)piperazine, N-(4-trifluoromethylphenyl)piperazine, 1-(4-bromophenyl)piperzine, and morpholine, depicted as follows:

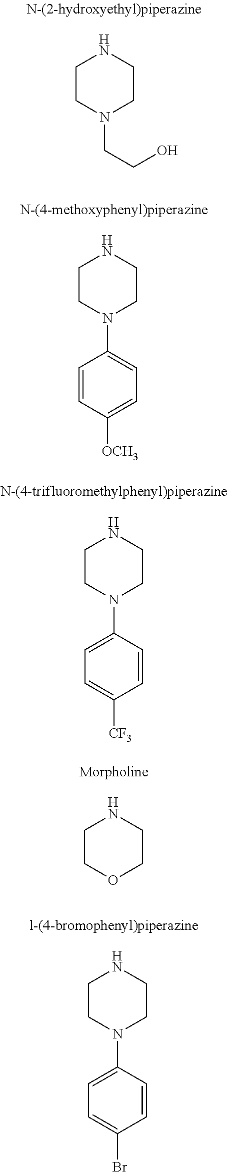 [SMILES: OCCN1CCNCC1 ; COc1ccc(N2CCNCC2)cc1 ; FC(F)(F)c1ccc(N2CCNCC2)cc1 ; C1COCCN1 ; Brc1ccc(N2CCNCC2)cc1]

Various other piperazine compounds suitable for reaction with a carboxylic acid/carboxylate group are commercially available.

Notably such amine compounds include a non-aromatic, or in other words aliphatic, cyclic group bearing a (e.g. secondary) amine group. In typical embodiments, the amine compound is a diamine. The second amine group may be a tertiary amine.

In other embodiments, such as morpholine, the compound is a monoamine. Morpholine is also illustrative of an aliphatic compound bearing an ether moiety.

Without intending to be bound by theory, it is surmised that the amide linking group may be involved in binding nucleic acids. Alternatively, the oxygen atoms of the aliphatic cyclic ring may also be involved. With reference to Example 18 of the forthcoming examples, when morpholine was reacted with the carboxylic acid groups or salts thereof on the surface of a solid support (e.g. particles), the resulting solid support (e.g. particles) had a zeta potential at a pH of 4.5 of −5.6 and a zeta potential of −37 at a pH of 8.5. This change in zeta potential is indicative of a change in the electrical potential near the electrical double layer. The electrical double layer includes any unreacted negative carboxylic acid/carboxylate surface charge of the particle and (e.g. positive) counterions in the solution that associate with the surface of the particle. Such change in electrical potential likely contributes to the ability of the ligand to bind and release DNA which can be beneficial for subsequent processing including size selection, amplification or modification of the DNA.

In other embodiments, the ligand further comprises an ionizable amine group. For example, when a diamine compound is reacted with the carboxylic acid groups or salts thereof on the surface of the solid support (e.g. particles), one of the amine groups forms an amide linkage and the other amine group of the ligand can reversibly ionize. The above diamine compounds and ligands formed from such compound each comprise a secondary or tertiary amine. It has been described in the literature that amine groups function as ionizable groups. Additionally, when piperazine molecules are reacted with carboxylic acids, the second nitrogen (that did not react to form an amide linkage) has a lowered pKa. This pKa depression allows effective elution of lower molecular weight nucleic acids using buffers that have pH values close to neutral (e.g. less than pH=9) to facilitate nucleic acid release under mild conditions. This is particularly useful as it can allow elution of specific nucleic acid fragments directly into buffers used in downstream processing step without the need for buffer exchange or desalting.

In other embodiments, carboxylic acid groups or salts thereof on the surface of the solid support (e.g. particles) are reacted with an amine compound comprising a fluorinated group.

In some embodiments, a representative solid support comprising a fluorinated ligand may be represented by the following formula:

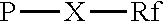

wherein P represents a solid support (e.g. particle);
X is an amide group; and
Rf is a monovalent fluorinated alkyl or ether group comprising no greater than 8 carbon atoms.

In this embodiment, the amine compound is a monoamine, such as 2,2,3,3,3-pentafluoropropylamine, depicted as follows:

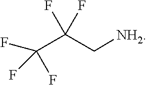

In other embodiments, a representative solid support comprising a fluorinated ligand may be represented by the following formula:

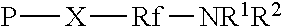

wherein P represents a solid support (e.g. particle);
X is an amide group; and
Rf is a divalent fluorinated alkyl or ether group comprising no greater than 8 carbon atoms; and
R′ is hydrogen and R² is hydrogen a C₁-C₄ alkyl group.

In this embodiment, the amine compound is a diamine, such as 2,2,3,3,5,5,6,6-octafluoro-4-oxa-heptan-1,7-diamine, depicted as follows:

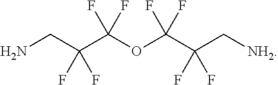 [SMILES: NCC(F)(F)C(F)(F)OC(F)(F)C(F)(F)CN]

Suitable fluorinated amine compounds have the formula:

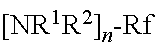

wherein Rf is a fluorinated hydrocarbon group optionally comprising an ether moiety;
R¹ and R² are independently hydrogen or a C₁-C⁴ alkyl group comprising no greater than 8 carbon atoms, and
n is 1 or 2.

In some embodiments, Rf of any of the above formulas comprises no greater than 6, 4, or 3 carbon atoms.

The amide linking group of the fluorinated amine ligands may function as a (e.g. non-covalent) binding group, as evidenced by the zeta potential. Without intending to be bound by theory, it is surmised that the amide linking group may be involved in binding nucleic acids with these ligands as well. With reference to Example 15 of the forthcoming examples, when 2,2,3,3,3-pentafluoropropylamine was reacted with the carboxylic acid groups or salts thereof on the surface of a solid support (e.g. particles), the resulting solid support (e.g. particles) had a zeta potential at a pH of 4.5 of 23.3 and a zeta potential of −25.1 at a pH of 8.5. This change in zeta potential is indicative of a change in the electrical potential near the electrical double layer. Such change in electrical potential likely contributes to the ability of the ligand to bind and release DNA which can be beneficial for subsequent processing including size selection, amplification or modification of the DNA.

Fluorinated amine ligands were also found to bind and release DNA as well as not inhibit enzymatic reactions that amplify DNA bound to the bead. Amine bonds (i.e. C—N) located next to a fluorocarbon bond (i.e. C—F) lowers the pKa of the amine nitrogen relative to the pKa of a non-fluorinate amine ligand. Similar to the amides from carboxylic acids or salts thereof and piperazine ligands, the lowering of the fluorinated amine pKa allows effective elution of lower molecular weight nucleic acids using buffers that have pH values close to neutral (e.g. less than pH=9) to facilitate nucleic acid release under mild conditions. This is particularly useful as it can allow elution of specific nucleic acid fragments directly into buffers used in downstream processing step without the need for buffer exchange or desalting.

Although the above solid support particle is depicted as having a single ligand, it is appreciated that the solid supports (e.g. each particle) comprises a plurality of ligands. For example, the solid support particles utilized in the examples are surmised to have approximately 0.6 mmol of carboxylic acid or carboxylate groups per gram of particles. Thus, if all such carboxylic acid or carboxylate groups were reacted with an amine compound as described herein, the number of ligands would be about equal to the number of carboxylic acid or carboxylate groups. When the particle is 1 um in diameter on average, the particles may contain up to approximately 1.8×10e-4 picomoles of ligand/μm². When the particle comprises a second ligand or unreacted carboxylic acid/carboxylate groups, the particles may contain lower amounts of ligand per surface area of the particle.

In some embodiments, the solid support (e.g. particles) comprising carboxylic acid or carboxylate groups is reacted with one amine compound, as described above, forming a first ligand. In other embodiments, the solid support (e.g. particles) is reacted with at least two different amine compounds, forming a first and second ligand. In other embodiments, the second amine compound may not function to bind polynucleic acids, but functions to simply lower the concentration of the first ligand. The weight ratio of the first amine compound to the second amine compound typically ranges from 1:10 to 10:1. Likewise, the weight ratio of first ligand to second ligand ranges from 1:10 to 10:1. In some embodiments, the weight ratio of the first amine compound or first ligand to second amine compound or ligand is at least 2:10, 3:10, 4:10, 5:10, 6:10, 7:10, 8:10, 9:10. In some embodiments, the weight ratio of the first amine compound or first ligand to second amine compound or ligand is no greater than 9:10, 8:10, 7:10, 6:10, 5:10, 4:10, 3:10, 2:10. Any combination of the described amine compounds can be utilized. In some embodiments, the first amine compound is piperazine or a derivative thereof and the second amine compound is morpholine. In other embodiments, the first amine compound is a fluorinated diamine and the second amine compound is a fluorinated monoamine. In other embodiments, the solid support (e.g. particles) may further comprise other functional groups or other ligands that lack an amide group. For example, the solid support (e.g. particles) may comprise some unreacted carboxylic acid or carboxylate groups. In other embodiments, the solid support (e.g. particles) may comprise a known ligand for polynucleic acid processing, such as 6-aminocaproic acid or histamine dihydrochloride.

The zeta potential of the solid support (e.g. particles) comprising the described ligands can be measured according to the test method in the examples. The solid support (e.g. particles) comprising the ligand has a negative or positive zeta potential in the presence of a low pH buffer. In some embodiments, the pH of the low pH buffer is at least 3.5, 4, or 4.5. The solid support (e.g. particles) comprising the ligand has a lower zeta potential in the presence of a high pH buffer than when in the presence of a low pH of buffer. In some embodiments, the pH of the high pH buffer is at least 5.5, 6, or 6.5. The absolute value of the difference between the zeta potential in the presence of a low pH buffer and the zeta potential in the presence of a high pH buffer is typically at least 10, 20, 30, 40 or 50 mV. In one embodiment, the absolute value of the difference between the zeta potential at a pH of 4.5 and a zeta potential at a pH of 8.5 is at least 10, 20, 30, 40 or 50 mV. The absolute value of the difference in pH between the low and high pH buffer is typically at least 2, 3, or 4. In typical embodiments, the solid support (e.g. particles) comprising the ligand has a lower zeta potential at a pH of 8.5 than at a pH of 4.5. In some embodiments, the buffers utilized to characterize the zeta potential of solid support (e.g. particles) comprising the described ligands are trishydroxymethylaminomethane (TRIS) and sodium acetate buffer.

The solid support (e.g. particles) comprising the described ligands and kits can be utilized in a variety of polynucleic acid processing techniques including for example size separation, purification, quantification, amplification, tagmentation, digestion and preparation of libraries (e.g. for nucleic acid sequencing).

In some embodiments, the method of processing polynucleic acid comprises the following steps:
a) providing a solid support comprising ligands with an amide group, as previously described;
b) exposing the solid support to polynucleic acid molecules in a buffer having a low pH (e.g. less than 5.5) to bind at least a portion of the polynucleic acid molecules to the ligands;
c) optionally exposing the solid support with bound polynucleic acid molecules to a buffer having a higher pH (e.g. greater than 6) to release a portion of the bound polynucleic acid molecules from the ligands of the solid support and optionally retaining a portion of the polynucleic acid molecules bound to the solid support;
d) optionally washing the solid support comprising the bound and/or retained bound polynucleic acid molecules (such as after step b) and or step c));
e) optionally preparing a suspension from the solid support comprising the retained bound polynucleic acid molecules; and
f) utilizing the released portion of bound polynucleic acid molecules of c), and/or the retained portion of the bound polynucleic acid molecules bound to the solid support or suspension thereof.

With reference to step b) the binding of polynucleic acids generally occurs in the presence of a low pH buffer. In some embodiments, the pH of the low pH buffer is at least 3.5, 4, 4.5, 5 or 5.5. In some embodiments, the pH of the low pH buffer is no greater than 5.5, 5, 4.5, 4, or 3.5. In some embodiments, the solid support is exposed to polynucleic acid molecules in a buffer having a low pH for 10 minutes at ambient temperature (e.g. 25° C.).

With reference to step c), releasing of polynucleic acids generally occurs in the presence of a high pH buffer. In some embodiments, the pH of the high pH buffer is at least 5.5, 6, 6.5, 7, 7.5, 8, 8.5, 9, 9.5 or 10. In some embodiments, the pH of the high pH buffer is no greater than 10, 9.5, 9, 8.5, 8, 7.5, 7, 6.5, or 6. As illustrated by Table 6, in some embodiments, the amount of released DNA is about the same (e.g. varies by no more than about 10%) for a pH ranging from 6 to 12, as well as any range within the pH range of 6 to 12. In some embodiments, the high pH buffer has a pH ranging from 7 to 8, 7 to 9, or 7 to 10. In some embodiments, the solid support is exposed to polynucleic acid molecules in a buffer having a high pH for 10 minutes at ambient temperature (e.g. 25° C.).

Various low and high pH biological buffers for use in the method and kit are known.

Some suitable buffers include for example citrate buffer (sodium citrate and citric acid monohydrate), acetate buffer, and TE buffer as further described in the examples; phosphate-buffered saline (PBS); N-2-acetamido-2-aminocthanesulfonic acid (ACES); N-2-acetamido-2-iminodiacetic acid (ADA); amino methyl propanediol (AMP); 3-1,1-dimethyl-2-hydroxyethylamino-2-hydroxy propanesulfonic acid (AMPSO); N,N-bis2-hydroxyethyl-2-aminocthanesulfonic acid (BES); N,N-bis-2-hydroxyethylglycine (BICINE); bis-2-hydroxyethyliminotrishydroxymethylmethane (Bis-Tris); 1,3-bistrishydroxymethylmethylaminopropane (BIS-TRIS Propane); 4-cyclohexylamino-1-butane sulfonic acid (CABS); 3-cyclohexylamino-1-propane sulfonic acid (CAPS); 3-cyclohexylamino-2-hydroxy-1-propane sulfonic acid (CAPSO); 2-N-cyclohexylaminoethanesulfonic acid (CHES); 3-N,N-bis-2-hydroxyethylamino-2-hydroxypropanesulfonic acid (DIPSO); N-2-hydroxyethylpiperazine-N-3-propanesulfonic acid (EPPS or HEPPS); N-2-hydroxyethylpiperazine-N-4-butanesulfonic acid (HEPBS); N-2-hydroxyethylpiperazine-N-2-ethanesulfonic acid (HEPES); N-2-hydroxyethylpiperazine-N-2-propanesulfonic acid (HEPPSO); 2-N-morpholinoethanesulfonic acid (MES); 4-N-morpholinobutanesulfonic acid (MOBS); 3-N-morpholinopropanesulfonic acid (MOPS); 3-N-morpholino-2-hydroxypropanesulfonic acid (MOPSO); piperazine-N—N-bis-2-ethanesulfonic acid (PIPES); piperazine-N—N-bis-2-hydroxypropanesulfonic acid (POPSO); N-trishydroxymethyl-methyl-4-aminobutanesulfonic acid (TABS); N-trishydroxymethyl-methyl-3-aminopropanesulfonic acid (TAPS); 3-N-trishydroxymethyl-methylamino-2-hydroxypropanesulfonic acid (TAPSO); N-trishydroxymethyl-methyl-2-aminoethanesulfonic acid (TES); N-trishydroxymethylmethylglycine (TRICINE); trishydroxymethylaminomethane (TRIS); histidine and polyhistidine; imidazole and derivatives thereof; triethanolamine dimers, oligomers and polymers; and di/tri/oligo amino acids, for example Gly-Gly; and Ser-Ser, Gly-Gly-Gly, and Ser-Gly.

In some embodiments, the low pH buffer is citrate buffer (sodium citrate and citric acid monohydrate) or acetate buffer. In some embodiments, the high pH buffer is PBS or TRIS.

The method may optionally comprise one or more washing steps. In one embodiment, the method comprises washing the solid support comprising the bound polynucleic acid molecules after step b). This washing step typically utilizes a low pH buffer. In some embodiments, this wash step utilizes the same buffer as step b). In another embodiment, the method comprises washing the solid support comprising the retained polynucleic acid molecules after step c). This washing step typically utilizes a high pH. Illustrative suspension buffers, also described as storage buffers, include PBS and TE buffer. In some embodiments, the suspension buffer has a pH of about 8. Neutral pH buffers, or water, can also be used.

The buffers generally have an ion salt concentration of less than about 1 M. In some embodiments, the salt concentration of the buffer during binding is less than 500 mM, 250 mM, 100 mM, 50 mM, 25 mM or 10 mM. A common suitable salt for use in (e.g. (poly)nucleic acid capture) buffers is sodium chloride. In one embodiment, the buffer has a pH greater than 6 and a salt concentration of less than 1M, 500 mM, 250 mM, 100 mM, 50 mM, 25 mM or 10 mM. In some embodiments, as illustrated by Table 5, the amount of bound DNA and released DNA is about the same (e.g. varies by no more than about 10%) for salt concentrations ranging from 10 to 500 mM.

In some embodiments, at least some of the method steps may be performed with a kit. The kit typically comprises the solid support comprising ligands and a low pH buffer (e.g. having a pH of less than 5.5), as previously described. In some embodiments, the kit further comprise a high pH buffer, as previously described suitable for releasing a portion of polynucleic acid molecules. In some embodiments, the kits further comprise a wash and/or suspension buffer, as previously described.

A variety of polynucleic acids can be processed using the solid supports comprising ligands, methods, and kits described herein. In some embodiments, the polynucleic acids comprise at least 100, 200, 300 400 of 500 base pairs. In some embodiments, the polynucleic acids comprise at least 1000, 1500, 2000 (e.g. 2027, 2322), 2500, 3000, 3500, 4000 (e.g. 4361), 4500, or 5000 base pairs. In some embodiments, the polynucleic acids comprise at least 5500, 6000, 6500 (e.g. 6557), 7000, 7500, 8000, 8500, 9000 (e.g. 9461), or 10,000 base pairs. In some embodiments, the polynucleic acids comprise at least 150,000; 20,000 (e.g. 23130), 25,000; 30,000; 35,000; 40,000; 45,000, 50,000 (e.g. 48502) base pairs or greater. In some embodiments, the polynucleic acid comprises a distribution of sizes having a minimum and maximum defined by an interval of the number of base pairs just described.

In some embodiments, the following performance criteria described herein were obtained with respect to a DNA standard or a mixture of standards, as described in greater detail in the forthcoming examples. Representative commercially available DNA standards include for example 2. DNA (i.e. duplex DNA isolated from bacteriophage lambda that is 48,502 base pairs in length), 2 DNA-HindIII digest (i.e. DNA isolated from bacteriophage lambda digested with the restriction endonuclease HindIII to produce 8 DNA fragments of sizes ranging from 125 bp to 23,130 bp), 1 kb DNA ladder (i.e. DNA fragments with a size range of 500 bp to 10 kb); 100 bp DNA ladder (i.e. DNA fragments with a size range of 100 bp to 1517 bp). Unless stated otherwise, the processing of a DNA standard was conducted at a ratio of polynucleic acids (e.g. DNA):solid support (e.g. particles) has a weight ratio of 1:250 w/w. Other ratios are also suitable. For example, the weight ratio of polynucleic acids (e.g. DNA):solid support (e.g. particles) may range from 1:10 to 1:2500. In some embodiments, the weight ratio of polynucleic acids (e.g. DNA):solid support (e.g. particles) is at least 1:25, 1:50, 1:100, 1:150, or 1:200. In some embodiments, the weight ratio of polynucleic acids (e.g. DNA):solid support (e.g. particles) is no greater than 1:2500; 1:2000; 1:1500, 1:1000, or 1:500.

In some embodiments, the size and distribution of the polynucleic acids is known, such in the case of the standards. In other embodiments, the size and distribution of the polynucleic acids can be determined with methods known in the art, such as pulsed-field gel electrophoresis.

Unless specified otherwise, the following terms are defined as follows:

“Supernatant” refers to the solution left behind after polynucleic acid molecules (e.g. DNA) are bound to the solid support (e.g. particles). Thus, characterization of supernatant pertains to the polynucleic acid molecules (e.g. DNA) that don't bind; Percent bound polynucleic acid molecules (e.g. DNA) can be calculated according to the formula 100×(Initial DNA ng−Supernatant DNA ng)/Initial DNA ng.

“Eluate” refers to the solution of polynucleic acids molecules (e.g. DNA) initially bound to the beads, but released when exposed to a higher pH buffer for 10 minutes at room temperature;

“Solid support (e.g. particle) suspension” refers to providing the solid support (e.g. particles) in an aqueous liquid after separation of eluate containing the released polynucleic acids molecules (e.g. DNA). The aqueous liquid may be characterized as a carrier liquid that conveys the solid support (e.g. particles) with the bound polynucleic acids to subsequent processing and analysis steps. Some of the polynucleic acids bound to the solid support (e.g. particles) may be released into the aqueous liquid of the suspension. However, in typical embodiments, the amount of polynucleic acids bond to the solid support (e.g. partilces) is significantly greater than the amount released into the aqueous liquid of the suspension. For example, the amount of polynucelic acids released into the aqueous liquid of the suspension may be less than 10, 5, or 1 wt. % as compared to the total amount of polynucelic acids of the suspension (i.e. the sum of polynucleic acids bound to the solid support and released into the aqueous liquid of the suspension).

In some embodiments, the solid support (e.g. particles), methods, and kits comprising the described ligands can be utilized to process (e.g. bind) polynucleic acids (e.g. DNA) ranging in size from 100 to 50,000 base pairs, such as 2 DNA and 2 DNA-HindIII Digest mix in an 80:20 v:v ratio.

In some embodiments, the DNA of the supernatant and eluate were quantified. The bound DNA was calculated as described above.

With reference to Table 3, in some embodiments, the unbound polynucleic acid molecules in the supernatant have a mass of polynucleic acid molecules less than 90, 80, 70, 60, 50, 40, 30, or 20% of the total initial polynucleic acid molecules. In some embodiments, the retained bound polynucleic acid molecules have a mass of polynucleic acid molecules greater than 10, 20, 30, 40, 50, 60, 70, 80, 90% of the total initial polynucleic acid molecules. In some embodiments, the mass of released polynucleic acid molecules is greater than 10, 20, 30, 40, 50, 60, 70, 80, 90% of the total initial polynucleic acid molecules. In some embodiments, the solid support comprises a greater amount of retained bound polynucleic acid molecules than released polynucleic acid molecules. For example, the mass of retained bound polynucleic acid molecules is greater than 50, 55, 60, 65, 70, 75, 80, 85, 90% and the mass of released polynucleic acid molecules is less than 50, 45, 40, 35, 30, 25, 20, 15, or 10%. In contrast, when the solid support (e.g. particles) comprised carboxylic acid/carboxylate (i.e. Comparative Example A) in the absence of the described ligands, the amount of bound polynucleic acid was less than 2% and no polynucleic acids were released.

In other embodiments, after steps a-c), the method further comprises quantifying the size and distribution of the polynucleic acids (e.g. DNA) fragment sizes with methods known in the art, such as pulsed-field gel electrophoresis. With reference to Table 4, the initial DNA (e.g. of the standard) contains a specific concentration of certain DNA fragments ranging in size from 125 base pairs to 48,505 base pairs. Notably, in the case of Comparative A, lacking the described ligand, no DNA was detected in the eluate. Thus, no DNA was released. When the amount of DNA of a particular size fragment is a lower relative concentration in the supernatant and eluate as compared to the initial DNA, it indicates that the DNA remaining bound to the bead has been enriched for that DNA fragment size. Notably, the amount of DNA in the supernatant and eluate for the 23,130 bp and 48,502 bp fragment sizes is less than the initial DNA. Notably, the solid support comprising ligands, methods, and kits described herein are advantageous for binding and retaining polynucleic acid molecules having greater than 10,000; 15,000, 20,000; 25,000; 30,000, 35,000, 40,000, 45,000, 50,000 or greater base pairs. The amount of bound retained DNA can be greater than 20, 30, 40, 50, 60, 70, 80, 90% of a fragment of a specific size (e.g. 23130 or 48502).

These results indicate that the steps of binding and releasing polynucleic acid molecules is amenable for size selection or size separation of polynucleic acids, especially higher molecular weight fragments. The ability to perform size selection by eluting a biased distribution of molecular weight fragments while still being able to utilize both populations (bound and eluted nucleic acid) was unexpected and beneficial because all of the nucleic molecules, whether they were eluted or not, are now accessible for utilization. This enables size selection with direct utilization of both populations without buffer exchange or desalting. Both populations can be utilized in the same way or taken on to different process steps. This also maximizes the usable and accessible nucleic acids after size selection because both populations can be utilized.

As illustrated by Table 7, high amounts of bound and released polynucleic acids, as previously described, can be achieved using other buffers (e.g. sodium acetate rather than citrate buffer). In contrast, when the solid support (e.g. particles) comprised carboxylic acid/carboxylate (i.e. Comparative Example A) in the absence of the described ligands, the amount of bound polynucleic acid was less than 10% and 4% of polynucleic acids were released.

With reference to Table 8, high amounts of bound and released polynucleic acids can be achieved with polynucleic acids (e.g. DNA) ranging in size from 100 to 1000 base pairs. In these embodiments, the amount of bound polynucleic acids (e.g. DNA) was at least 50, 60, 70, 80, 90 or even 100%. Further, the amount released was at least 50, 60, 70, or 80%.

In some embodiments, the solid support (e.g. particles), methods, and kits comprising the described ligands can be utilized for amplifying polynucleic acids. Numerous techniques are available for amplifying nucleic acids. These techniques include polymerase chain reaction (PCR), ligase chain reaction (LCR), self-sustained sequence replication (3SR), nucleic-acid-sequence-based amplification (NASBA), strand displacement amplification (SDA), transcription-mediated isothermal CR cycling probe technology, cascade rolling circle amplification (CRCA), nicking endonuclease amplification reaction (NEAR), transcription mediated amplification (TMA), loop-mediated isothermal amplification (LAMP), helicase-dependent amplification (HDA), CRISPR-Cas-based amplification, and in vitro transcription (IVT).

PCR (Polymerase Chain Reaction) is a method for amplifying a target DNA sequence using a heat-stable DNA polymerase and two nucleotide primers, one complementary to the (+)-strand at one end of the sequence to be amplified and the other complementary to the (−)-strand at the other end. Because the newly synthesized DNA strands can subsequently serve as additional templates for the same primer sequences, successive rounds of primer annealing, strand elongation, and dissociation produce rapid and highly specific amplification of the desired sequence.

Rolling Circle Amplification (RCA) is an amplification process driven by a DNA polymerase which can replicate with either linear or geometric kinetics under isothermal (single temperature) conditions. In the presence of two suitably designed primers, a geometric amplification occurs via DNA strand displacement and hyperbranching to generate 10¹² or more copies of DNA template in 1 hour.

With reference to Table 9, the amount of the retained bound polynucleic acid molecules (e.g.). DNA) may be greater than 50, 60, 70, 80, 90% of the total initial polynucleic acid molecules. In many embodiments, the amount of retained bound polynucleic acid molecules is greater than Comparative Examples A and C. In some embodiments, the amount of released polynucleic acid molecules have a mass of less than 15, 14, 13, 12, 11, 10, 9, 8, 7, 6, 5, 4, 3, 2, or 1%. In some embodiments, the retained bound polynucleic acid molecules have a mass of less than 50% when the ligands were derived from piperazine, N-methyl piperazine, or N-phenylpiperazine.

With reference to Table 10, Ct values lower than those of the initial DNA demonstrate greater (e.g. PCR) amplification. Notably, a difference of 6 in a Ct value is generally equivalent to a 100-fold difference in the amount of the target polynucleic acid. Thus, even small reductions in Ct values are of significance. In some embodiments, the reduction in Ct values is at least 1, 2, 3, 4, 5, 6, 7, 8, 9, or 10, as compared to buffer lacking polynucleic acid. Reduction in Ct values are evident in the eluate as well as the bead suspension at dilutions of 1:10 and 1:100. In many instances the Ct values are less than the Ct values obtained with Comparative Example A and C. Thus, the solid support (e.g. particles), methods, and kits comprising the described ligands are advantageous for amplifying higher molecular weight fragments, such as the 23 kb HindIII digest fragment of λ DNA. In some embodiments, the reduction in Ct values for the bead suspension is greater than the reduction in Ct values for the eluate.

With reference to Table 11, the amount of retained bound polynucleic acid molecules (e.g. 2. DNA-HindIII digest) may be greater than 50, 55, 60, 65, 70, 75, 80, 85, 90% of the total initial polynucleic acid molecules. In many embodiments, the amount of retained bound polynucleic acid molecules is greater than Comparative Examples A and C. In some embodiments, the amount of released polynucleic acid molecules has a mass of less than 50, 45, 40, 35, 30, 25, 20, or 15%.

With reference to Table 12, Ct values lower than those of amplified input DNA solution demonstrates greater (e.g. PCR) amplification, as previously described. In some embodiments, the reduction in Ct values is at least 1, 2, 3, 4, or 5. In some embodiments, significant reductions in Ct values are evident using bead suspensions, especially at a dilution of 1:10. In many instances the Ct values are less than the Ct values obtained with Comparative Example A and C. Thus, the solid support (e.g. particles), methods, and kits comprising the described ligands are also suitable and can be advantageous for amplifying lower molecular weight fragments, such as the 4 kb-HindIII digest fragment of 2. DNA.

With reference to Table 13, the amount of amplified DNA from the bead suspension may be greater than the amplified input DNA solution. In many embodiments, the amount of amplified DNA is greater than Comparative Examples A and C. Thus, polynucleic acids were also successfully amplified using rolling circle amplification.

The solid support (e.g. particles) comprising the described ligands, methods, and kits can be utilized for library preparation for nucleic acid sequencing. A sequencing library is a collection of DNA fragments that have been modified with nucleic acid adaptors into a format that is compatible with the sequencing technology and instrument under use. Each sequencing instrument has its own library preparation workflows to add the necessary barcodes and adaptors and modify the fragments to enable sequencing. Libraries can be made for high-throughput sequencing instruments, which rely on methods such as sequencing by synthesis, sequencing by ligation, sequencing by binding, pyrosequencing or impedance-based sequencing, or real-time long-read instruments which rely on methods such as sequencing by synthesis in zero-mode waveguide or nanopore sequencing. In some embodiments, this sequencing includes tagmentation and enzymatic reactions to fragment the DNA. In some embodiments, the enzyme may be bonded to a bead such as exemplified by bead-linked transposomes of the “Illumina DNA Prep” reference guide.

With reference to Table 14, in some embodiments, the final DNA concentrations of a (e.g. 16S) sequencing library were at least 50, 75, 100, 125, 150, 175, 200, 225, 250, 275, or 300 ng/μL. In some embodiments, the suspension was undiluted. In other embodiments, the suspension was diluted at concentrations of 1:10 or 1:100. In many instances the final DNA concentrations were greater than Comparative Examples A and C. Embodiments with the greatest final concentrations are typically preferred.

The solid support (e.g. particles) comprising the described ligands, methods, and kits can be utilized for shotgun metagenomic sequencing library preparation from bead-isolated HindIII digested Lambda DNA from the bead suspensions. With reference to Table 15, negative numbers are indicative of a lack of enrichment of a HindIII digest fragment size; whereas high positive numbers are indicative of enrichment for a HindIII digest fragment size. The eluate of Examples 2 and 3 had greater enrichments than Comparative Example C for fragments of 6.557 kilo base pairs, 4.351 kilo base pairs, 2.322 kilo base pairs and 0.125 kilo base pairs or in other words for polynucleic acids ranging in size from 0.125 kilo base pairs to 6.557 kilo base pairs.

The solid support (e.g. particles) comprising the described ligands, methods, and kits can be utilized for shotgun metagenomic sequencing library preparation from bead-isolated microbial genomic DNA (gDNA) from the bead suspensions. With reference to Table 16, the relative abundance of DNA in a standard comprised of gDNA from different bacteria (e.g. Bacillus, Enterococcus, Escherichia, Lactobacillus, Listeria, Pseudomonas, Salmonella, Staphylococcus) was similar for the DNA standard and the bead suspensions of Examples 2 and 3. To quantify the difference observed between theoretical and the DNA standard used directly, the Bray-Curtis distances (dissimilarity) were calculated using R package Vogan (version 2.5-7) and reported in Table 17. The distance between aDNA standard alone and bead suspension was less than the DNA standard to the theoretical distribution, indicating less error was introduced through associating the DNA to the beads than through the library prep and sequencing method itself.

Utilizing the released and/or retained and/or suspended polynucleic acid molecules may comprise processing or analyzing the polynucleic acids including for example size separation, purification, quantification, detection or modification (e.g. via exposure to at least one enzyme) such as amplification, transcription, tagmentation, digestion, ligation, or preparation of libraries (e.g. for nucleic acid sequencing).

In view of the favorable test results obtained by testing of DNA standard, it is surmised that the solid support (e.g. particles) comprising the described ligands, methods, and kits can be utilized for polynucleic acids extracted from any suitable living source, including human, animal, microbial, plant or viral sources, including cells, saliva, fresh tissue or other materials containing DNA, whether initially whole or otherwise wholly or partially disrupted. In some embodiments, the solid support (e.g. particles) comprising the described ligands, methods, and kits can be utilized for testing for target DNA, such as from microbes, or samples of food.

Various polynucleic acid molecules can be processed including bacteria and those obtained from microbes, fungi, plants, or animals. A biological sample comprising polynucleic acids can be a naturally-occurring sample or deliberately designed or synthesized sample or library. In one embodiment, the sample contains a population of cells or cell fragments, including without limitation cell membrane components, exosomes, and sub-cellular components. The cells may be a homogenous population of cells, such as isolated cells of a particular type, or a mixture of different cell types, such as from a biological fluid or tissue of a human or mammalian or other species subject. The biological sample can be simple, for example containing isolated DNA or deriving from a homogeneous cell culture or tissue source, or can be complex, such as deriving from tumor, blood, or whole organ samples. The biological sample can be from any suitable source, such as a healthy tissue or cell source, a diseased tissue or cell source, a cell culture or line, cell extracts or lysates, a biopsy, and the like.

Other biological samples comprising polynucleic acids include for use include blood samples, including serum, plasma, whole blood, and peripheral blood, saliva, urine, vaginal or cervical secretions, amniotic fluid, placental fluid, cerebrospinal fluid, or serous fluids, mucosal secretions (e.g., buccal, vaginal, or rectal). Still other samples include a blood-derived or biopsy-derived biological sample of tissue or a cell lysate (i.e., a mixture derived from tissue and/or cells). Other suitable tissues include hair, fingernails, and the like. Additional samples include libraries of antibodies, antibody fragments and antibody mimetics like affibodies. Other samples can be synthesized or engineered collections of chemical molecules, proteins, antibodies or any other of the polyanions described herein.

### EXAMPLES

Unless otherwise noted, all parts, percentages, ratios, etc. in the Examples and the rest of the specification are by weight. Unless otherwise indicated, all other reagents were obtained, or are available from fine chemical vendors such as MilliporeSigma, Burlington, Massachusetts, or may be synthesized by known methods. Table 1 (below) lists materials used in the examples and their sources.

TABLE 1
Materials List

| DESIGNATION | DESCRIPTION | SOURCE |
|---|---|---|
| SBead | carboxylate-modified magnetic | Millipore SigmaBurlington, |
| | particles (hydrophilic) obtained | MA |
| | under the trade name “SERA- | |
| | MAG SPEEDBEAD”, product | |
| | number 45152105050250 | |
| DBead | magnetic beads with surface | Thermo Fisher Scientific |
| | carboxylic acid groups obtained | Inc., Waltham, MA |
| | under the trade name “MyOne ™ | |
| | DYNABEADS ™”, product | |
| | number 65012 | |
| CSwitch | Magnetic beads with the trade | Thermo Fisher Scientific |
| | name “CHARGESWITCH ™” | Inc. |
| | obtained as a component of the | |
| | ChargeSwitch ™ gDNA Mini | |
| | Bacteria Kit, kit product number | |
| | CS11301 | |
| Comparative Example D | magnetic beads with surface | Thermo Fisher Scientific |
| DBead-amine | amino groups obtained under the | Inc., Waltham, MA |
| | trade name “Dynabeads ™ M-270 | |
| | Amine”, product number 14308D | |
| AAH | Acetic anhydride | MilliporeSigma |
| DMF | N,N-dimethylformamide | Millipore Sigma |
| DIEA | Diisopropyl ethyl amine | Millipore Sigma |
| TEA | Triethylamine | Millipore Sigma |

Buffers

| MES (pH 6) | Morpholinoethylsulfonate 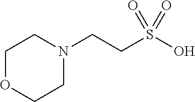 [SMILES: O=S(=O)(O)CCN1CCOCC1] | EMD Biosciences, Inc., La Jolla, CA |
|---|---|---|
| Sodium citrate | Sodium citrate 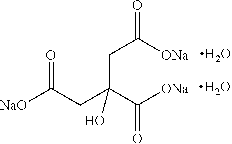 | EMD Chemicals, Inc (Gibbstown, NJ) |
| Citric acid monohydrate | Citric acid monohydrate | Alfa Aesar, Ward Hill, MA |
| | 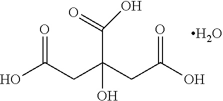 | |
| Citrate buffer (pH 4) | Aqueous citrate buffer solution at pH 4-Prepared by mixing | |
| | aqueous solutions of sodium citrate and citric acid monohydrate. | |
| NaCl | Sodium chloride | EMD Millipore |
| | | Corporation, Billerica, |
| | | MA |
| Tris buffer (pH 7.5) | 1M aqueous tris(hydroxymethyl)- | Invitrogen, Grand Island, |
| | aminomethane hydrochloride | NY |
| Tris elution buffer (pH 8.5) | 10 mM Tris/10 mM NaCl | |
| PBS (pH 7.4) | Phosphate-buffered saline | Invitrogen |
| | solution (concentration: 1×) | |
| Acetate buffer (pH 4.5) | 3M sodium acetate, pH 4.5 (aqueous) | Thermo Fisher Scientific, Inc. |
| | 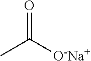 | |
| TE buffer (pH 8) | Buffer with 10 mM Tris, 1 mM | Invitrogen |
| | EDTA | |
| EDC | N-ethyl-N′-(3-dimethylaminopropyl) | Oakwood Chemical, |
| | carbodiimide hydrochloride | Estill, SC |
| UltraPure DNase/RNase-Free | Molecular biology grade water, | Invitrogen |
| Distilled Water | product number 10977015 | |

Amine Compounds

| | | |
|---|---|---|
| Piperazine | Piperazine | Alfa Aesar |
| N-methylpiperazine | N-methylpiperazine | Millipore Sigma |
| N-(hydroxyethyl)piperazine | N-(2-hydroxyethyl)piperazine | Millipore Sigma |
| N-(4-methoxyphenyl)piperazine | N-(4-methoxyphenyl)piperazine | MilliporeSigma |
| N-(4-trifluoromethylphenyl) | N-(4-trifluoromethylphenyl) | MilliporeSigma |
| piperazine | piperazine | |
| Morpholine | Morpholine | Alfa Aesar |
| Fluorinated diamine | 2,2,3,3,5,5,6,6-octafluoro-4-oxa- | |
| | heptan-1,7-diamine, synthesized | |
| | as described in Preparative | |
| | Example 1 (PE-1) of | |
| | WO2020035776A1 | |
| Fluorinated monoamine PFPA | 2,2,3,3,3-pentafluoropropylamine | MilliporeSigma |

Polynucleic Acid (DNA) Samples

| | | |
|---|---|---|
| λ DNA | Duplex DNA isolated from | New England Biolabs, |
| | bacteriophage lambda. Lambda | Ipswich, MA |
| | DNA is 48,502 base pairs | |
| | in length, product number N3011 | |
| λ DNA-HindIII digest | HindIII-digest of lambda DNA | New England Biolabs |
| | with 8 fragments having size | |
| | range: 125 bp to 23,130 bp, | |
| | product number N3012 | |
| 1 kb DNA ladder | 1 kilo-base pair (kb) DNA ladder | New England Biolabs |
| | (Size range: 500 bp to 10 kb), | |
| | product number B7025 | |
| 100 bp DNA ladder | 100 base pair (bp) DNA ladder | New England Biolabs |
| | (Size range: 100 bp-1,517 bp), | |
| | product number N3231 | |
| ZymoBIOMICS Microbial | Defined mixture of microbial | Zymo Research, Irvine CA |
| Community DNA Standard | genomic DNA from 10 species, | |
| | product number D6305 | |
| Qubit dsDNA HS Assay Kit | QUBIT assay kit for dsDNA, | Invitrogen |
| | product number Q32854 | |
| Qubit 1X dsDNA HS Assay | QUBIT assay kit for dsDNA, | Invitrogen |
| Kit | product number Q33231 | |
| Vortexer | Obtained as VORTEX-GENIE 2 | Scientific Industries, |
| | mixer | Bohemia, NY |
| Microcentrifuge tubes | 1.5 ml low adhesion | USA Scientific, Ocala, FL |
| | microcentrifuge tubes, | |
| | polypropylene | |
| SYBR Green PCR Master Mix | qPCR mastermix used in Lambda | Thermo Fisher |
| | DNA Sybr qPCR test method, | |
| | product number 4364344 | |
| Agilent Brilliant III Master | qPCR mastermix used in E. coli | Agilent |
| Mix | Genomic DNA qPCR test | |
| | method, product number 600888 | |
| EQUIPHI29 DNA Polymerase | Kit for rolling circle | Thermo Fisher |
| Kit | amplification, product number | |
| | A39390 | |
| Exo-resistant random primers | Random DNA heximers with | Thermo Fisher |
| | modification to provide | |
| | resistance to exonucleases, | |
| | product number SO181 | |
| dNTPs | combination of four nucleotides, | Invitrogen |
| | for PCR applications, product | |
| | number 18427088 | |
| Nextera XT Index Kit v2 set A, | Unique indexes to add to DNA | Illumina, San Diego, CA |
| and set B | libraries to facilitate | |
| | multiplexing, product number | |
| | FC-131-2001 (set A) and FC- | |
| | 131-2002 (set B) | |
| Illumina DNA Prep Library | Kit to produce shotgun | Illumina |
| Preparation kit | metagenomics sequencing | |
| | libraries, product number | |
| | 20018704 | |
| Illumina MiSeq v3 Reagent | Reagent consumables required | Illumina |
| Cartridge | for sequencing on the Illumina | |
| | MiSeq, product number MS-102- | |
| | 3003 | |

### GENERAL PROCEDURES

### General Procedure A: Passivation of Residual Positive Charge on Speedbeads

Procedure adapted from passivation strategy detailed in U.S. Patent Publication 20190071662A1. 2 mL of SBead solution (100 mg beads) was added to a 50 mL Falcon tube and diluted with 8 mL water. The beads were magnetically isolated, and the supernatant was discarded. The isolated beads were washed with 2×10 mL DMF and the supernatant was discarded after each wash. The beads were resuspended in 7.8 mL DMF, and 2 mL AAH and 200 μL DIEA were added. The tube was shaken at 250 revolutions per minute (rpm) for 30 minutes (min). The beads were isolated over 10 min and the brown supernatant was discarded. Then the beads were washed with 3×10 mL DMF and finally resuspended in 2 mL DMF for storage.

### General Procedure B: Aqueous Functionalization of Magnetic Beads

500 μL Tris buffer (pH=8.5, 10 mM) was added to 0.2 mL of the passivated SBead stock solution from General Procedure A (10 mg, 5.8 micrometer (μm) active functionality) or 1000 μL DynaBeads were added directly to a 1.5 mL Eppendorf tube. The solution was mixed, the beads were isolated, and the supernatant was discarded. The beads were washed with 3×500 μL MES buffer (pH=6, 25 mM) and isolated. The beads were resuspended in 100 μL total of stock solutions of piperazine derivatives and morpholine (each stock solution is 120 micromole/milliliter (μmol/mL) in MES buffer, ratio of the two solutions is adjusted to tune the ratio of acyl piperazine to morpholine on the final beads, 12 μmol total) and mixed for 30 minutes. 229 μL of freshly prepared EDC solution at 0° C. (10 mg/mL in MES buffer, 12 μmol) and 4.3 μL fresh MES buffer were added and the reaction was mixed at room temperature overnight. The beads were isolated and washed with 2×500 μL Tris buffer (pH=7.5, 50 mM) and 2×500 μL PBS. The beads were left in 1000 μL PBS (10 mg/mL) and stored at 4° C. until further use.

### General Procedure C: Functionalization of Passivated Speedbeads in DMF

0.2 mL of the passivated SBead stock solution from General Procedure A (10 mg, 5.8 μm active functionality) was added to a 1.5 mL Eppendorf tube and the beads were isolated. 300 μL TEA solution (5.5 mg/mL in DMF, 16.2 μmol) and 229 μL EDC solution (10 mg/mL in DMF, 12 μmol) were added to the beads and mixed for 5 min. 100 μL total of stock solutions of piperazine derivatives and morpholine was added (each stock solution is 120 μmol/mL in DMF, the ratio of the two solutions was adjusted to tune the ratio of acyl piperazine to morpholine on the final beads, 12 μmol total) and the solution was mixed at room temperature overnight. The beads were isolated and washed with 2×500 μL Tris buffer and 2×500 μL PBS. The beads were left in 1000 μL PBS (10 mg/mL) and stored at 4° C. until further use.

### General Procedure D: Binding of DNA onto Beads

Functionalized magnetic beads (25 μL) of Table 2 were added to the bottom of a non-binding 96-well plate (Greiner Bio-One, Frickenhausen, Germany). The binding buffer (75 μL), 10 mM citrate buffer at pH 4, was added to the well and mixed thoroughly. The plate was placed on a plate magnet (Invitrogen, Waltham, MA) for 2 min. The liquid was removed, then DNA (specific DNA indicated in each example) and 10 mM citrate pH 4 buffer was added to the well. The beads were resuspended by aspirating with a pipette and then left to sit at room temperature (RT) for 10 min. After placing on a plate magnet, the residual liquid was collected as the supernatant. Wash buffer (citrate buffer pH 4 at 10 mM (100 μL)) was added to the wells and the beads were resuspended in the liquid by aspirating with a pipette. After placing the plate on the magnet, the residual liquid was removed. The elution buffer (100 μL), 10 mM Tris/10 mM NaCl pH 8.5, was added and the beads were resuspended by pipette aspiration. After letting sit at RT for 10 min, beads were separated from the liquid using the plate magnet and the liquid was collected as eluate. Separated beads were resuspended in 100 μL storage solution (TE buffer) as bead suspension. If needed, samples were stored at −20° C. until further use.

### Test Methods

### Zeta Potential Measurements

Zeta potential measurements were taken on a Malvern Zetasizer Nano ZSP (Malvern Panalytical, Malvern, United Kingdom). 2.5 microliters (μL) of a 10 milligram/milliliter (mg/mL) solution of functionalized beads from General Procedures B or C was added to 5 mL acetate buffer (pH=4.5, 10 millimolar (mM)) or Tris buffer (pH=8.5, 10 mM) and loaded onto a disposable folded capillary cell (part #DTS1070). The cell was equilibrated at room temperature for 60 seconds(s) and then analyzed with the Smoluchowski approximation in the Zetasizer instrument software. Three measurements were taken and averaged, with each measurement having a minimum of 10 and maximum of 100 runs.

### DNA Quantification Test Method

Qubit assay kits were used as instructed. An example for an experiment analyzing 18 samples with 2 standards is detailed here. All assay components were equilibrated to room temperature. A stock solution of “working solution” was prepared by mixing 20 μL of “reagent” with 3980 μL of “buffer” and vortexed on low speed to ensure complete mixing. For each sample, 10 μL was mixed with 190 μL of working solution in Qubit tubes and vortexed. Standards were prepared by mixing 10 μL of the 2 standards with 190 μL of the working solution in a Qubit tube, then vortexed. The tubes were then read individually in the Qubit fluorometer, which reports the concentration of analyte in the sample. Based on this concentration and final volume of the sample, the amount of DNA bound and recovered was calculated and reported in this report as %. When the DNA concentration was too low for detection in the Qubit fluorometer, the DNA % is reported as 0.

### DNA Fragment Analysis Test Method

Solutions of DNA were characterized using an automated pulsed-field capillary electrophoresis system, the Femto Pulse System from Agilent Technologies, Inc. (Santa Clara, CA). The system and kit were used as instructed. The electropherogram was integrated at the indicated DNA fragment size representative of the DNA mixture used in the Examples. The relative peak areas between the fragment sizes are reported as %.

qPCR

Dilutions (1:10, 1:100) of retained bead samples, eluate, supernatant, and wash solutions were prepared in molecular grade water using low-bind 1.5 mL microcentrifuge tubes (4043-1021, USA Scientific, Ocala, FL). Two microliters of each sample (undiluted, 1:10 diluted, 1:100 diluted) were tested in duplicate by qPCR. The qPCR mix contained the following in a final volume of 25 μL: 1×SYBR Green PCR Master Mix (Applied Biosystems by Thermo Fisher Scientific (4309155, Waltham, MA)), 0.5 μM forward primer Lambda1F 5′-CGG CGT CAA AAA GAA CTT CC-3′, and 0.5 μM reverse primer Lambda1R 5′-GCA TCC TGA ATG CAG CCA TA-3′. Primers were synthesized by Integrated DNA Technologies (Coralville, IA). The qPCR was run in a skirted PCR plate (Agilent 401490) sealed with optically-clear strip caps (401425, Agilent Technologies, Santa Clara, CA) using an Agilent AriaMx instrument with the following parameters: 10 min at 95° C., 40 cycles of 15 seconds at 95° C. and 1 min at 60° C. followed by melt analysis: 30 seconds at 95° C., 30 seconds at 65° C.-95° cat 0.5° C. increments.

Lambda DNA Sybr qPCR Test Method

The qPCR standard was prepared by making 10-fold dilutions of Lambda DNA 500 μg/mL (New England Biolabs, #N3011) in molecular grade water (Invitrogen, #10977015). The 1:100 dilution (5 ng/μL) was used as the high standard concentration which contained 1.91×10⁸ Lambda DNA copies in 5 μL. A total of 7 standard dilutions were run, down to 5 femtograms/microliter (fg/μL) (191 copies in 5 μL), included a no-template control (NTC). Samples from DNA capture processing (bead suspensions and eluates) were all diluted 10-fold and 100-fold in molecular grade water. The qPCR reactions were prepared with SYBR™ Green PCR Master Mix (Thermo Fisher, #4364344), with a final concentration of 0.625 micromolar (μM) F and 0.625 UM R primers and 5 μL of sample template or DNA standard. The PCR cycles were as follows: 10 min at 95° C., followed by 40 cycles of 15 seconds at 95° C. and 1 min at 60° C. Primers were used that targeted the 23 kb fragment (Lambda1F: CGG CGT CAA AAA GAA CTT CC, Lambda1R: CAG TCA ACC ACC AGG GAA TAA) and the 4 kb fragment Lambda 4 kb F: TGG CAT TCT GGA GGG AAA TAC, Lambda 4 kb R: CAG TCA ACC ACC AGG GAA TAA) of HindIII digested Lambda DNA.

E. coli Genomic DNA 16S qPCR Test Method

16S qPCR was performed using Agilent Brilliant III master mix (#600888) and primers and probes described in the literature (Suzuki et al. “Quantitative analysis of small-subunit rRNA genes in mixed microbial populations via 5′-nuclease assays”, Applied and Environmental Microbiology, 2000; 66 (11),4605-14.) Primer and probe final concentrations and sequences used were as follows: BACT1369F: CGGTGAATACGTTCYCGG 1.5 uM, PROK1492R: GGWTACCTTGTTACGACTT 1 uM, TAQMAN MGB Probe (Thermo Fisher) TM1389F: 5′FAM-CTTGTACACACCGCCCGTC-3′MGB-NFQ 0.5 μM. PCR cycling conditions were as follows: 10 min at 95° C., 40 cycles of 95° C. for 15 seconds and 57° C. for 1 min.

### Rolling Circle Amplification Test Method

Rolling circle amplification was performed using the EQUIPHI29 DNA Polymerase Kit (Thermo Fisher, #A39390), exo-resistant random primers (Thermo Fisher, #SO181), and dNTP Mix 10 mM (Invitrogen, #18427088) following manufacturer's instructions. Lambda DNA was bound onto beads following the protocol listed before. One (1) microliter of bead suspension was used for the DNA template. Reactions were incubated at 45° C. and sampled after 30 min, 1 hour, and 2 hours and DNA amplification was assessed by quantifying DNA using QUBIT 1×dsDNA HS Assay Kit and QUBIT reader (Thermo Fisher).

### 16S Library Preparation

16S metagenomics libraries were prepared following the Illumina (San Diego, CA) 16S Sample Preparation Guide (Part #15044223 Rev. B). Amplicon primers used targeted the 16S V3 and V4 regions as described in the literature (Klindworth et al. “Evaluation of general 16S ribosomal RNA gene PCR primers for classical and next-generation sequencing-based diversity studies” Nucleic Acids Research, 2013, 41 (1), e1). 16S Amplicon PCR Forward Primer: TCGTCGGCAGCGTCAGATGTGTATAAGAGACAGCCTACGGGNGGCWGCAG; 16S Amplicon PCR Reverse Primer: GTCTCGTGGGCTCGGAGATGTGTATAAGAGACAGGACTACHVGGGTATCTAATCC. Index PCR was performed with Illumina NEXTERA XT Index Kit v2 set A (FC-131-2001). Individual sample libraries were quantified with the QUBIT dsDNA HS Assay Kit (Thermo Fisher, Q32854).

### Shotgun Metagenomics Library Preparation and Sequencing

Shotgun metagenomics libraries were prepared from the bead suspension of ZymoBIOMICS Microbial Community DNA Standard (Zymo Research, Irvine, CA, #D6305) extracted with the same beads as above; the ZymoBIOMICS DNA standard was also used directly in library preparation (positive control).

Libraries were prepared following the Illumina DNA Prep Library Preparation kit, using Illumina bead-linked transposome technology (Illumina, 20018704) following manufacture's protocols (1000000025416 v09) with the following specifications and modifications:
(a) Samples were quantified with QUBIT 1×dsDNA HS Assay Kit and Qubit reader, and diluted to contain 30 ng DNA in 30 μL sample; this was used as input to the library preparation protocol.
(b) Samples were given unique barcodes prior to multiplexing, using the NEXTERA XT Index Kit v2 Set B (Illumina, FC-131-2002)
(c) Final libraries were diluted to 12 μM loading concentration, and loaded onto an Illumina MiSeq v3 Reagent Cartridge (600 cycle; Illumina, MS-102-3003), followed by 2×150 sequencing.

Reads derived from Lambda DNA were aligned to the Lambda genome (New England Biolabs, Ipswich, MA) using bowtie2 (v. 2.3.5.1) (Langmead et al. “Fast gapped-read alignment with Bowtie 2”, Nature Methods, 2012, 9, 357-359). To enable visualization and calculations of read depth across HindIII fragments, the following steps were taken to convert files: .sam alignment files output from bowtie2 were converted to .bam, sorted, and indexed using samtools (v. 1.11). Sorted .bam files were converted to .bedgraph using bamCoverage, normalizing for library depth using CPM method (deeptools v. 3.5.1) (Ramirez et al. “deepTools 2: A next-generation web server for deep-sequencing data analysis”, Nucleic Acids Research, 2016, 44 (1), W160-W165); the genome together with these bedgraphs were used to visualize read coverage in IGV (v. 2.8.13) (Robinson et al. “Integrative genomics viewer”, Nature Biotechnology, 2011, 29 (1), 24-26). To calculate mean coverage over each HindIII-digest of Lambda DNA genome fragments, a .bed file containing the locations of HindIII cut sites in the New England Biolabs Lambda genome was created, and the bedtools map function was used to sum read coverage over each HindIII-derived fragment (specifying -o sum and -c 4, as files were .bedgraph and not .bed) (v. 2.30.0) (Quinlan et al. “BEDTools: a flexible suite of utilities for comparing genomic features”, Bioinformatics, 2010, 26 (6), 841-842). Mean read counts for each HindIII fragment were calculated for the initial DNA sample. Then, the log 2 ratios of sample read counts to initial HindIII DNA sample readcounts on a per-fragment basis were calculated.

Reads derived from the ZymoBIOMICS DNA standard were taxonomically classified with Kraken2 (v. 2.0.8-beta) (Wood et al. “Improved metagenomic analysis with Kraken 2”, Genome Biology, 2019, 20 (1), 257) using the standard database followed by quantitation with Bracken (v. 2.0) (Lu et al. “Bracken: estimating species abundance in metagenomics data”, PeerJ (San Francisco, CA), 2017, 1, e104). All pairwise distances were computed between samples (Bray-Curtis distance generated with R (v.4.1.0) package Vegan (v. 2.5-7)).

### EXAMPLES

### Example 1 to Example 19 and Comparative Examples A to C

Several samples of magnetic beads were functionalized with ligands according to General Procedures A, B, or C. These functionalized beads were designated as Examples 1 to 18. Two samples of magnetic beads were not functionalized with ligands and were designated as Comparative Examples A and B. Commercially available Cswitch beads were designated as Comparative Example C. Commercially available Comparative Example D beads are described above. Examples 1 to 19 and Comparative Examples A to C and D had concentrations of 10 mg/mL. Details on the general functionalization procedures used, the ligands employed, and the zeta potentials of the resulting bead samples are found in Table 2.

TABLE 2
Magnetic beads generated and corresponding Zeta Potentials in various buffers

| | | | | Co-ligand | | | |
|---|---|---|---|---|---|---|---|
| | | | | (in feed) | | ZP [mV] | |
| | General | Magnetic | | Morpholine, | PFPA, | pH = | pH = |
| Sample | Procedure | Bead | Ligand | mol % | mol % | 4.5 | 8.5 |
| Comparative | A | SBead | None | None | None | −8.4 | −37.1 |
| Example A | | | | | | | |
| Comparative | n/a | DBead | None | None | none | −34.5 | −38.0 |
| Example B | | | | | | | |
| Comparative | n/a | CSwitch | None | None | None | | |
| Example C | | | | | | | |
| Example 1 | B | SBead | Piperazine | 75 | | 2.3 | −37.2 |
| Example 2 | B | SBead | Piperazine | 0 | | 16.4 | −37.6 |
| Example 3 | B | SBead | N-methylpiperazine | 0 | | 18.9 | −35.5 |
| Example 4 | B | SBead | N-(hydroxyethyl) | 0 | | 20.0 | −35.9 |
| | | | piperazine | | | | |
| Example 5 | B | SBead | N-phenylpiperazine | 0 | | −5.8 | −42.6 |
| Example 6 | B | SBead | N-(4-methoxyphenyl) | 0 | | −7.8 | −40.8 |
| | | | piperazine | | | | |
| Example 7 | C | SBead | N-(4- | 0 | | −0.47 | −31.3 |
| | | | trifluoromethylphenyl) | | | | |
| | | | piperazine | | | | |
| Example 8 | B | DBead | Piperazine | 0 | | 26.9 | −20.2 |
| Example 9 | B | DBead | Piperazine | 50 | | 20.9 | −27.5 |
| Example 10 | B | DBead | Piperazine | 75 | | 11.9 | −34.0 |
| Example 11 | B | DBead | Piperazine | 90 | | 5.5 | −32.8 |
| Example 12 | B | SBead | Fluorinated diamine | | 90 | 23.9 | −33.1 |
| Example 13 | B | SBead | Fluorinated diamine | | 75 | 28.7 | −30.8 |
| Example 14 | B | SBead | Fluorinated diamine | | 50 | 26.7 | −34.8 |
| Example 15 | B | SBead | Fluorinated diamine | | 0 | 23.3 | −25.1 |
| Example 16 | B | SBead | N-methylpiperazine | 80 | | 4.5 | −36.6 |
| Example 17 | B | SBead | N-methylpiperazine | 90 | | −12.1 | −36 |
| Example 18 | B | SBead | Morpholine | 100 | | −5.6 | −37 |
| Example 19 | B | SBead | 2,2,3,3,3- | None | 100 | NM | NM |
| | | | pentafluoropropyl | | | | |
| | | | Amine (PFPA) | | | | |

NM = not measured

Binding of DNA (λ DNA and λ DNA-HindIII Digest mix in an 80:20 v:v ratio) onto magnetic beads prepared in previous Examples was carried out according to General Procedure D. The average initial amount of DNA was close to 1 μg, see table for exact DNA mass, and the DNA:bead ratio was 1:250 w/w for this example. The DNA in the supernatant and eluate were quantified using DNA quantification test method and further characterized using pulsed-field capillary electrophoreses (using a Femto Pulse system, Agilent Technologies) to characterize the DNA fragments sizes. The results of these analyses are shown in Tables 3 and 4.

TABLE 3
DNA bound to the beads released in eluate

| | Initial | Supernatant | DNA | DNA |
|---|---|---|---|---|
| Magnetic Beads | DNA, ng | DNA, ng | bound, % | released, % |
| Comparative | 919 | 903 | 1.70 | 0 |
| Example A | | | | |
| Comparative | 881 | 864 | 1.89 | 0 |
| Example B | | | | |
| Example 1 | 864 | 157 | 81.8 | 40.0 |
| Example 2 | 864 | 107 | 87.7 | 38.3 |
| Example 5 | 919 | 210 | 77.1 | 41.8 |
| Example 6 | 919 | 449 | 51.1 | 15.8 |
| Example 7 | 919 | 391 | 57.4 | 26.7 |
| Example 10 | 881 | 287 | 67.4 | 34.9 |
| Example 11 | 881 | 312 | 64.6 | 39.6 |
| Example 12 | 919 | 99.9 | 89.1 | 23.5 |
| Example 13 | 919 | 204 | 77.8 | 18.4 |
| Example 14 | 919 | 374 | 59.3 | 14.4 |
| Example 15 | 919 | 301 | 67.2 | 10.0 |
| Example 19 | 597 | 385 | 34.0 | 8.0 |
| Comparative | 597 | 196 | 66.3 | 0 |
| Example D | | | | |

Values in this table represent the average values from three replicates.

TABLE 4
DNA fragment analysis of DNA (in supernatant and eluate)

| | | Rel. concentration (%) at each size | | | | | | | | |
|---|---|---|---|---|---|---|---|---|---|---|
| | | Size, bp | | | | | | | | |
| | Solution | 125 | 564 | 2027 | 2322 | 4361 | 6557 | 9416 | 23130 | 48502 |
| Initial DNA | | 0.1 | 0.8 | 3.6 | 3.8 | 8.5 | 12.6 | 19.6 | 44.7 | 6.3 |
| Example 1 | Supernatant | 0 | 0 | 0.8 | 1.3 | 6.6 | 15.3 | 35.6 | 40.2 | 0.1 |
| | Eluate | 0.3 | 1.4 | 5.6 | 6.8 | 13.5 | 20.5 | 31.9 | 19.7 | 0.2 |
| Example 2 | Supernatant | 0.1 | 0.1 | 1.6 | 2.4 | 8.4 | 18.2 | 37.6 | 31.4 | 0.2 |
| | Eluate | 0.5 | 1.5 | 5.8 | 7.7 | 13.3 | 22.3 | 29.6 | 19.1 | 0.1 |
| Comparative | Supernatant | 1.5 | 2.5 | 8.3 | 10.5 | 16.8 | 21.6 | 21.6 | 17 | 0.3 |
| Example A | Eluate | ND | ND | ND | ND | ND | ND | ND | ND | ND |
| Example 5 | Supernatant | 0 | 0 | 0.9 | 1 | 5.1 | 15.1 | 23.8 | 53.7 | 0.3 |
| | Eluate | 0.3 | 1.7 | 7.4 | 7.2 | 15.1 | 20 | 24.7 | 23.6 | 0.1 |
| Example 6 | Supernatant | 0 | 0 | 2.4 | 2.4 | 8.4 | 16.3 | 23.4 | 47 | 0.1 |
| | Eluate | 0.6 | 3.1 | 9.3 | 9.2 | 14.6 | 17.2 | 35.7 | 10.2 | 0.1 |
| Example 7 | Supernatant | 0 | 0 | 1.1 | 1.3 | 5.8 | 14 | 21.3 | 55.9 | 0.6 |
| | Eluate | 0.5 | 2.4 | 9 | 9.3 | 16.2 | 19 | 23.3 | 20.1 | 0.1 |
| Example 12 | Supernatant | 0 | 0.1 | 1.6 | 1.9 | 9.1 | 16.7 | 24.7 | 45.7 | 0.3 |
| | Eluate | 0.6 | 3.3 | 12 | 11.6 | 17.5 | 19.4 | 28.6 | 6.9 | 0 |
| Example 13 | Supernatant | 0 | 0.1 | 2.1 | 2.6 | 9.2 | 15.9 | 23.7 | 46.1 | 0.4 |
| | Eluate | 0.7 | 3.6 | 12.5 | 12 | 17.4 | 18.9 | 27.6 | 7.3 | 0 |
| Example 14 | Supernatant | 0.2 | 0.2 | 1.7 | 2 | 8.2 | 16.2 | 25.8 | 45.6 | 0 |
| | Eluate | 0.9 | 4.7 | 13.4 | 13.8 | 18.1 | 19.3 | 22.3 | 7.4 | 0 |
| Example 15 | Supernatant | 0 | 0.1 | 2.4 | 2.9 | 8.9 | 15.1 | 22 | 47.7 | 0.8 |
| | Eluate | 1 | 3.3 | 6.4 | 10 | 13.5 | 20.3 | 27.8 | 17.5 | 0.1 |
| Example 19 | Supernatant | 0 | 0 | 2.3 | 2.9 | 8.8 | 15.1 | 22.2 | 47.6 | 1.1 |
| | Eluate | 1.6 | 5.6 | 10.2 | 11.2 | 15.8 | 15.5 | 28.6 | 11.7 | 0 |

ND = no DNA detected.

Binding of DNA (λ DNA and λ DNA-HindIII Digest mix in an 80:20 v:v ratio) onto magnetic beads prepared in previous examples was carried out as described in General Procedure D. The sodium chloride concentration in the elution buffer was systematically varied, and the results are displayed in Table 5. The average initial amount of DNA was 1 μg and the DNA:bead ratio was 1:250 w/w for this example.

TABLE 5
Quantified DNA bound at pH 4 and released
at pH 8.5 with indicated NaCl concentration

| Functionalized | Average % | Elution NaCl | Average % |
|---|---|---|---|
| Magnetic Beads | bound | concentration(mM) | released |
| Comparative | 4.75 | 10 | 0 |
| Example A | | | |
| Comparative | 4.37 | 500 | 0 |
| Example A | | | |
| Example 5 | 92.6 | 10 | 45.8 |
| Example 5 | 88.8 | 500 | 51.0 |
| Example 3 | 83.3 | 10 | 21.7 |
| Example 3 | 95.2 | 500 | 24.2 |

Values in this table represent the average values from three replicates.

Binding of DNA (λ DNA and 2 DNA-HindIII Digest mix in an 80:20 v:v ratio) onto magnetic beads prepared in previous Examples was carried out as described in General Procedure D. The binding buffer, 10 mM sodium acetate buffer at pH 4.5, was used in place of citrate buffer. The average initial amount of DNA was 1 μg and the DNA:bead ratio was 1:250 w/w for this example. The results are in Table 6.

TABLE 6
Quantified DNA released at various pH solutions

| Elution buffer | Average % released |
|---|---|
| 10 mM sodium phosphate/NaCl pH 6 | 6.45 |
| 10 mM sodium phosphate/NaCl pH 6.5 | 8.63 |
| 10 mM sodium phosphate/NaCl pH 7 | 8.77 |
| 10 mM sodium phosphate/NaCl pH 7.5 | 12.4 |
| 10 mM sodium phosphate/NaCl pH 8 | 10.7 |
| 10 mM sodium phosphate/NaCl pH 8.7 | 8.95 |
| 10 mM Tris HCl/NaCl pH 7.5 | 6.43 |
| 10 mM Tris HCl/NaCl pH 8.0 | 8.31 |
| 10 mM Tris base/NaCl pH 10 | 11.6 |

Values in this table represent the average values from three replicates.

Binding of DNA (λ DNA and λ DNA-HindIII Digest mix in an 80:20 v:v ratio) onto magnetic beads prepared in previous Examples was carried out as described in General Procedure D. The binding buffer, 10 mM sodium acetate buffer at pH 4.5, was used in place of citrate buffer. The average initial amount of DNA was 1.04 μg and the DNA:bead ratio was 1:250 w/w for this example. The results are in Table 7.

TABLE 7
Quantified DNA bound at pH 4.5 and released at pH 8.5

| Functionalized | Supernatant | Average % | Average % |
|---|---|---|---|
| Magnetic Beads | DNA (ng) | bound | released |
| Comparative Example A | 943 | 9.34 | 4.02 |
| Example 2 | 268 | 74.1 | 20.7 |
| Example 15 | 201 | 80.5 | 12.0 |
| Example 3 | 156 | 84.9 | 19.9 |
| Example 5 | 352 | 66.0 | 19.2 |
| Example 4 | 299 | 71.1 | 15.1 |

Values in this table represent the average values from three replicates.

Binding of DNA (100 base pair (bp) ladder and 1 kilo-base pair (kb) ladder mix in a 50:50 v:v ratio) onto magnetic beads prepared in previous Examples was carried out as described in General Procedure D. The average initial amount of DNA was close to 1 ug and the DNA:bead ratio was 1:250 w/w for this example. The results are in Table 8.

TABLE 8
Quantified DNA bound at pH 4 and released at pH 8.5

| | Average % | Average % |
|---|---|---|
| Functionalized Magnetic Beads | bound | released |
| Comparative Example A | 0 | 0 |
| Example 2 | 100 | 77.1 |
| Example 3 | 100 | 67.6 |
| Example 4 | 100 | 75.6 |

Values in this table represent the average values from three replicates.

Starting with λ DNA, DNA was captured onto functionalized magnetic beads prepared in previous Examples according to General Procedure D. The amounts of DNA bound and released are shown in Table 9. The Ct values from SYBR qPCR targeting the 23 kb λ DNA-HindIII digest fragment of the bead suspension and eluate diluted 1:10 and 1:100 are reported in Table 10.

TABLE 9
Quantified DNA bound at pH 4.5 and released at pH 8.5

| Magnetic | Initial | DNA bound, % | | DNA released, % | |
|---|---|---|---|---|---|
| Beads | DNA, ng | Set #1 | Set #2 | Set #1 | Set #2 |
| Comparative | 768 | 49.1 | 34.4 | 10.1 | 9.4 |
| Example C | | | | | |
| Comparative | 768 | 2.2 | 7.2 | 0.0 | 0.0 |
| Example A | | | | | |
| Example 16 | 768 | 50.3 | 58.2 | 3.0 | 2.7 |
| Example 17 | 768 | 60.0 | 67.8 | 4.5 | 3.1 |
| Example 18 | 768 | 63.4 | 63.4 | 5.0 | 3.5 |
| Example 12 | 768 | 63.7 | 60.2 | 2.3 | 2.6 |
| Example 13 | 768 | 54.3 | 42.4 | 1.6 | 1.6 |
| Example 14 | 768 | 43.4 | 32.7 | 0.8 | 1.5 |
| Example 15 | 768 | 59.0 | 62.6 | 1.2 | 2.0 |
| Example 4 | 768 | 58.6 | 67.2 | 2.6 | 2.5 |
| Example 5 | 768 | 25.0 | 29.8 | 3.9 | 3.6 |
| Example 6 | 768 | 54.0 | 51.0 | 11.2 | 10.5 |
| Example 1 | 768 | 54.0 | 61.7 | 5.6 | 5.0 |
| Example 7 | 768 | 73.4 | 74.9 | 8.2 | 6.8 |
| Example 2 | 768 | 38.5 | 32.2 | 2.0 | 1.6 |
| Example 3 | 768 | 43.6 | 49.5 | 1.9 | 2.7 |

TABLE 10A
SYBR qPCR results targeting the 23 kb HindIII digest fragment
of λ DNA of eluate and bead suspension diluted 1:10 and 1:100.

| | Ct (cycle number) | | | | Ct (cycle number) | | | |
|---|---|---|---|---|---|---|---|---|
| | of 1:10 dilution | | | | of 1:100 dilution | | | |
| Sample | Avg | SD | | | Avg | SD | | |
| Initial DNA | 15.22 | 0.42 | | | 18.70 | 8.71 | | |
| | | | Bead | | | | Bead | |
| | Eluate | | suspension | | Eluate | | suspension | |
| Sample | Avg | SD | Avg | SD | Avg | SD | Avg | SD |
| Comparative | 20.12 | 0.43 | 19.99 | 0.64 | 21.87 | 0.57 | 20.33 | 0.32 |
| Example C | | | | | | | | |
| Comparative | 18.90 | 0.14 | 18.84 | 0.48 | 22.25 | 0.23 | 23.04 | 0.53 |
| Example A | | | | | | | | |
| Example 16 | 15.39 | 0.26 | 15.08 | 2.39 | 18.08 | 0.21 | 17.68 | 2.76 |
| Example 17 | 14.84 | 0.12 | 18.26 | 4.36 | 17.80 | 0.20 | 15.55 | 0.64 |
| Example 18 | 14.17 | 0.29 | 12.94 | 0.25 | 17.73 | 0.52 | 15.06 | 0.21 |
| Example 12 | 15.47 | 0.03 | 14.11 | 0.98 | 18.32 | 0.32 | 14.32 | 0.74 |
| Example 13 | 16.33 | 0.08 | 12.43 | 0.07 | 19.02 | 0.41 | 15.88 | 0.61 |
| Example 14 | 16.48 | 0.21 | 13.01 | 0.89 | 19.14 | 0.22 | 17.45 | 0.47 |
| Example 15 | 16.88 | 0.06 | 12.10 | 0.60 | 19.69 | 0.55 | 16.94 | 1.86 |
| Example 4 | 15.61 | 0.26 | 11.83 | 0.54 | 18.72 | 0.58 | 14.94 | 1.17 |
| Example 5 | 15.37 | 0.12 | ND | ND | 18.32 | 0.24 | 17.44 | 0.33 |
| Example 6 | 14.44 | 0.31 | ND | ND | 16.69 | 0.45 | 16.38 | 0.11 |
| Example 1 | 15.20 | 0.26 | 13.12 | 0.21 | 18.13 | 0.20 | 15.55 | 0.67 |
| Example 7 | 13.99 | 0.04 | 22.54 | 10.31 | 16.80 | 0.21 | 15.67 | 0.25 |
| Example 2 | 16.03 | 0.13 | 12.22 | 0.30 | 19.30 | 0.38 | 17.06 | 0.53 |
| Example 3 | 15.97 | 0.19 | 12.32 | 0.63 | 19.00 | 0.29 | 16.06 | 0.34 |

TABLE 10B
SYBR qPCR results targeting the 23 kb HindIII digest fragment
of λ DNA of eluate and bead suspension diluted 1:10 and 1:100.

| | Ct (cycle number) | | | | Ct (cycle number) | | | |
|---|---|---|---|---|---|---|---|---|
| | of 1:10 dilution | | | | of 1:100 dilution | | | |
| Sample | Avg | SD | | | Avg | SD | | |
| Initial DNA | 12.13 | 0.16 | | | 15.42 | 0.42 | | |
| | | | Bead | | | | Bead | |
| | Eluate | | suspension | | Eluate | | suspension | |
| Sample | Avg | SD | Avg | SD | Avg | SD | Avg | SD |
| Comparative | 24.05 | 0.46 | 24.14 | 0.10 | 26.15 | 0.31 | 25.28 | 2.39 |
| Example D | | | | | | | | |
| Example 19 | ND | ND | 16.05 | 0.15 | 18.4 | 1.23 | 19.43 | 0.29 |

Starting with λ DNA-HindIII digest, DNA was captured onto magnetic beads prepared in previous Examples as described in General Procedure D. The total dsDNA in supernatant and eluate was detected and percent bound and released was calculated (Table 11). The Ct values from SYBR qPCR targeting the 4 kb λ DNA-HindIII digest fragment of the bead suspension and eluate diluted 1:10 and 1:100 are reported in Table 12.

TABLE 11
Total DNA (ng) remaining in supernatant and released in eluate.
SYBR qPCR targeting the 4 kb HindIII digest fragment in
eluate and bead suspension diluted 1:10 and 1:100.

| | Initial | DNA bound, % | | DNA released, % | |
|---|---|---|---|---|---|
| Sample | DNA, ng | Set #1 | Set #2 | Set #1 | Set #2 |
| Comparative | 576 | 71.2 | 66.3 | 50.2 | 78.0 |
| Example C | | | | | |
| Comparative | 576 | 1.4 | 7.8 | 2.4 | 2.9 |
| Example A | | | | | |
| Example 16 | 576 | 89.5 | 87.6 | 26.6 | 40.8 |
| Example 17 | 576 | 82.5 | 90.0 | 28.5 | 44.3 |
| Example 18 | 576 | 88.2 | 91.3 | 47.4 | 47.6 |
| Example 12 | 576 | 95.1 | 99.0 | 41.3 | 57.6 |
| Example 13 | 576 | 85.6 | 92.1 | 33.0 | 36.5 |
| Example 14 | 576 | 75.5 | 85.8 | 20.1 | 27.1 |
| Example 15 | 576 | 83.4 | 94.4 | 17.5 | 24.0 |
| Example 4 | 576 | 83.4 | 91.7 | 29.7 | 35.9 |
| Example 5 | 576 | 48.1 | 58.7 | 20.8 | 26.0 |
| Example 6 | 576 | 66.1 | 73.3 | 35.2 | 40.3 |
| Example 1 | 576 | 92.9 | 97.9 | 38.0 | 60.4 |
| Example 7 | 576 | 75.0 | 79.5 | 27.8 | 33.9 |
| Example 2 | 576 | 60.9 | 70.1 | 26.2 | 30.0 |
| Example 3 | 576 | 76.4 | 86.2 | 30.6 | 34.7 |

TABLE 12A
SYBR qPCR targeting the 4 kb λ DNA-HindIII digest fragment of λ
DNA of eluate and bead suspension diluted 1:10 and 1:100

| | Ct (cycle number) | | | | Ct (cycle number) | | | |
|---|---|---|---|---|---|---|---|---|
| | of 1:10 dilution | | | | of 1:100 dilution | | | |
| Sample | Avg | SD | | | Avg | SD | | |
| Initial DNA | 11.01 | 0.09 | | | 15.61 | 0.26 | | |
| | | | Bead | | | | Bead | |
| | Eluate | | suspension | | Eluate | | suspension | |
| Sample | Avg | SD | Avg | SD | Avg | SD | Avg | SD |
| Comparative | 10.20 | 0.15 | 13.24 | 0.70 | 14.11 | 0.28 | 15.91 | 0.33 |
| Example C | | | | | | | | |
| Comparative | 14.79 | 0.29 | 15.83 | 0.52 | 18.13 | 0.34 | 22.13 | 0.30 |
| Example A | | | | | | | | |
| Example 16 | 12.22 | 0.65 | 21.35 | 1.15 | 15.27 | 0.75 | 16.19 | 0.52 |
| Example 17 | 10.36 | 0.53 | 29.97 | 8.77 | 14.48 | 0.58 | 16.06 | 0.35 |
| Example 18 | 10.18 | 0.16 | 27.88 | 4.77 | 14.87 | 0.18 | 14.57 | 0.15 |
| Example 12 | 10.15 | 0.30 | 23.51 | 6.74 | 13.89 | 0.29 | 15.39 | 1.33 |
| Example 13 | 10.61 | 0.36 | 11.03 | 0.30 | 14.88 | 0.44 | 16.66 | 0.51 |
| Example 14 | 10.86 | 0.45 | 10.76 | 0.11 | 14.93 | 0.22 | 17.41 | 0.44 |
| Example 15 | 11.34 | 0.28 | 11.40 | 0.30 | 15.76 | 0.26 | 14.41 | 0.87 |
| Example 4 | 10.10 | 0.13 | 13.62 | 1.11 | 14.60 | 0.16 | 14.60 | 1.40 |
| Example 5 | 10.96 | 0.73 | ND | ND | 15.11 | 0.48 | 16.54 | 0.56 |
| Example 6 | 11.15 | 1.07 | ND | ND | 14.65 | 0.30 | 16.22 | 0.81 |
| Example 1 | 10.79 | 0.51 | 17.39 | 15.17 | 14.09 | 0.18 | 15.58 | 0.34 |
| Example 7 | 10.54 | 0.22 | 7.56 | 15.13 | 14.77 | 0.14 | 16.65 | 0.48 |
| Example 2 | 10.21 | 0.16 | 11.15 | 0.80 | 14.87 | 0.23 | 14.68 | 0.97 |
| Example 3 | 10.29 | 0.19 | 10.89 | 0.47 | 14.40 | 0.38 | 13.80 | 0.21 |

TABLE 12B
SYBR qPCR targeting the 4 kb λ DNA-HindIII digest fragment of λ
DNA of eluate and bead suspension diluted 1:10 and 1:100

| | Ct (cycle number) | | | | Ct (cycle number) | | | |
|---|---|---|---|---|---|---|---|---|
| | of 1:10 dilution | | | | of 1:100 dilution | | | |
| Sample | Avg | SD | | | Avg | SD | | |
| Initial DNA | 12.19 | 1.8 | | | 15.65 | 1.60 | | |
| | | | Bead | | | | Bead | |
| | Eluate | | suspension | | Eluate | | suspension | |
| Sample | Avg | SD | Avg | SD | Avg | SD | Avg | SD |
| Comparative | 21.24 | 0.30 | 26.38 | 0.69 | 24.88 | 0.46 | 29.72 | 0.92 |
| Example D | | | | | | | | |
| Example 19 | ND | ND | 18.70 | 0.48 | 21.40 | 2.05 | 21.65 | 2.64 |

Starting with Escherichia coli genomic DNA, DNA was captured onto functionalized magnetic beads prepared in previous Examples according to General Procedure D. Results were similar to SYBR qPCR targeting the 23 kb λ DNA-HindIII digest fragment of full-length Lambda DNA.

Starting with λ DNA, DNA was captured onto magnetic beads prepared in previous Examples using General Procedure D. The DNA quantities after rolling circle amplification from bead suspension diluted 1:100 are reported in Table 13.

TABLE 13
DNA quantity after rolling circle amplification
of λ DNA on beads in bead suspension solution

| | DNA concentration, ng/μL | | | | | |
|---|---|---|---|---|---|---|
| | | | 1:10 dilution | | 1:100 dilution | |
| | Undiluted sample | | Set | Set | Set | Set |
| Sample | Set #1 | Set #2 | #1 | #2 | #1 | #2 |
| Initial DNA | 415 | 413 | 218 | 232 | 146 | 92.1 |
| Comparative | Too High | Too High | 28.6 | 510 | 194 | 217 |
| Example C | | | | | | |
| Comparative | 217 | 193 | 228 | 140 | 183 | 93.8 |
| Example A | | | | | | |
| Example 16 | 345 | 325 | 223 | 70.8 | 108 | 50.2 |
| Example 17 | 348 | 358 | 213 | 262 | 95 | 77.8 |
| Example 18 | 252 | 436 | 139 | 178 | 5.41 | 4.88 |
| Example 12 | 211 | 186 | 260 | 253 | 103 | 140 |
| Example 13 | 540 | 397 | 310 | 273 | 286 | 117 |
| Example 14 | 324 | 388 | 303 | 260 | 157 | 133 |
| Example 15 | 490 | Too High | 369 | 368 | 222 | 218 |
| Example 4 | Too High | Too High | 370 | 570 | 254 | 167 |
| Example 5 | 364 | 407 | 374 | 485 | 292 | 253 |
| Example 6 | 398 | 440 | 388 | 335 | 177 | 146 |
| Example 1 | Too High | Too High | 325 | 318 | 179 | 211 |
| Example 7 | 378 | 447 | 423 | 339 | 193 | 174 |
| Example 2 | Too High | Too High | 302 | 245 | 147 | 142 |
| Example 3 | 442 | 403 | 282 | 358 | 133 | 120 |

Starting with Escherichia coli genomic DNA, DNA was bound to magnetic beads prepared in previous Examples using General Procedure D. The DNA concentration of the library after 16S library preparation of the bead suspensions undiluted, diluted 1:10, and diluted 1:100 are reported in Table 14.

TABLE 14
Final DNA concentrations of library after 16S library preparation
with bead suspension undiluted, diluted 1:10, and diluted 1:100

Final Library, ng/μL

| Functionalized Beads | Undiluted | 1:10 | 1:100 |
|---|---|---|---|
| Example 13 | 219 | 139 | 71.2 |
| Example 5 | 219 | 149.4 | 80.3 |
| Example 3 | 208 | 283 | 122 |
| Comparative Example C | 185 | 240 | 91.2 |
| Example 2 | 182 | 188 | 121 |
| Example 4 | 167 | 205 | 65 |
| Example 1 | 151 | 93.7 | 145 |
| Example 12 | 139 | 145 | 96.8 |
| Example 6 | 135 | 251 | 106 |
| Example 14 | 130 | 113 | 82.5 |
| Example 7 | 125 | 93.1 | 79.4 |
| Comparative Example A | 124 | 15.2 | 0.86 |
| Example 15 | 122 | 126 | 95.6 |
| Example 18 | 43.7 | 307 | 93.6 |
| Example 3 | 40 | 122 | 140 |
| Example 14 | 1.52 | 110 | 119 |
| Example 15 | 0.178 | 227 | 125 |

Electrophoresis of 16S sequencing libraries prepared from bead-isolated E. coli gDNA showed that libraries generated from functionalized beads produced the target molecular weight of ˜630 bp for Examples 1-7 and 12-18

Binding of DNA (λ DNA and λ DNA-HindIII Digest mix in an 80:20 v:v ratio) onto magnetic beads prepared in previous Examples was carried out as described in General Procedure D. Shotgun metagenomic libraries were prepared from bead suspensions. They were loaded onto a MISeq for sequencing. The Lambda-derived reads are shown in Table 15.

TABLE 15
Log₂ ratios of sample read counts/initial DNA read counts on a per fragment basis

| | | | | | log₂ (sample read counts/initial | | | |
|---|---|---|---|---|---|---|---|---|
| | | | | | HindIII-digested Lambda read counts) | | | |
| Size, kb | | 23.13 | 9.416 | 6.557 | 4.361 | 2.322 | 2.027 | 0.125 |
| Example 2 | Eluate | −1.57 | 0.01 | 0.48 | 0.96 | 3.21 | 1.94 | 1.72 |
| | Bead | 0.22 | −0.25 | −0.41 | −0.49 | 0.75 | −0.63 | −0.14 |
| | suspension | | | | | | | |
| Example 3 | Eluate | −1.78 | 0.03 | 0.52 | 1.02 | 3.27 | 2.00 | 1.91 |
| | Bead | 0.16 | −0.15 | −0.29 | −0.38 | 0.82 | −0.57 | −0.11 |
| | suspension | | | | | | | |
| Comparative | Eluate | −0.58 | 0.05 | 0.26 | 0.52 | 2.46 | 1.15 | 0.99 |
| Example C | Bead | 0.39 | −0.45 | −0.77 | −0.94 | 0.31 | −1.11 | −0.48 |
| | suspension | | | | | | | |

Starting with ZymoBIOMICS Microbial Community DNA Standard (Zymo Research, D6306), binding of DNA standards onto magnetic beads prepared in previous Examples was carried out as described in General Procedure D. The average initial amount of DNA was 500 ng and the DNA:bead ratio was 1:250 w/w. Shotgun metagenomic libraries were prepared from bead suspensions. The relative abundances of reads across DNA standard bacterial members are shown in Table 16 (below). The Bray-Curtis distances (dissimilarity) between Sample and the DNA standard used directly in library preparation and the theoretical (expected) distribution of the standard is shown in Table 17 (below).

TABLE 16
Relative abundance of reads across DNA standard bacterial members.
Theoretical represents the expected proportions of each genera of bacteria
assuming zero bias in extraction, library preparation, and sequencing.
DNA standard are samples in which the DNA standard was used directly
in sequencing without extraction.

| | Relative Abundance | | | | | | | | |
|---|---|---|---|---|---|---|---|---|---|
| Genus | | Bacillus | Enterococcus | Escherichia | Lactobacillus | Listeria | Pseudomonas | Salmonella | Staphylococcus |
| Theoretical | | 0.103 | 0.146 | 0.085 | 0.216 | 0.139 | 0.061 | 0.087 | 0.152 |
| DNA | Set #1 | 0.136 | 0.131 | 0.106 | 0.108 | 0.135 | 0.090 | 0.148 | 0.134 |
| Standard | Set #2 | 0.137 | 0.132 | 0.108 | 0.107 | 0.137 | 0.089 | 0.145 | 0.135 |
| Example 3 | Set #1 | 0.132 | 0.132 | 0.104 | 0.117 | 0.136 | 0.106 | 0.144 | 0.118 |
| | Set #2 | 0.130 | 0.130 | 0.102 | 0.118 | 0.136 | 0.110 | 0.147 | 0.116 |
| Example 2 | Set #1 | 0.129 | 0.130 | 0.102 | 0.126 | 0.135 | 0.105 | 0.142 | 0.121 |
| | Set #2 | 0.128 | 0.130 | 0.103 | 0.128 | 0.135 | 0.106 | 0.141 | 0.119 |
| Comparative | Set #1 | 0.129 | 0.126 | 0.102 | 0.124 | 0.130 | 0.076 | 0.139 | 0.165 |
| Example A | Set #2 | 0.121 | 0.116 | 0.101 | 0.135 | 0.122 | 0.093 | 0.142 | 0.159 |

TABLE 17
Bray-Curtis distances between Sample and (a) DNA standard
used directly in library prep or (b) the theoretical
(expected) distribution of the standard.

| | Average distance | Average distance to |
|---|---|---|
| Sample | to DNA standard | theoretical distribution |
| DNA Standard | 0.005 | 0.145 |
| Example 2 | 0.038 | 0.144 |
| Example 3 | 0.028 | 0.155 |

All cited references, patents, and patent applications in the above application for letters patent are herein incorporated by reference in their entirety in a consistent manner. In the event of inconsistencies or contradictions between portions of the incorporated references and this application, the information in the preceding description shall control. The preceding description, given in order to enable one of ordinary skill in the art to practice the claimed disclosure, is not to be construed as limiting the scope of the disclosure, which is defined by the claims and all equivalents thereto.

1. A solid support comprising ligands bound to the solid support; wherein at least a portion of the ligands comprises an amide group and a fluorinated amine group or a heterocyclic aliphatic group.

2. The solid support of claim 1 wherein the fluorinated amine group or a heterocyclic aliphatic group is an ionizable group.

3. The solid support of claim 1 wherein the solid support comprising ligands has a negative or positive zeta potential at a pH of 4.5.

4. The solid support of claim 1 wherein the solid support comprising ligands has a lower zeta potential at a pH of 8.5 than at a pH of 4.5.

5. The solid support of claim 4 wherein the absolute value of the difference between the zeta potential at a pH of 4.5 and a zeta potential at a pH of 8.5 is at least 10, 20, 30, 40 or 50 mV.

6. The solid support of claim 1 wherein the heterocyclic aliphatic group comprises an amine or ether moiety.

7. The solid support of claim 1 wherein the amide group is the reaction product of an acidic group or a salt thereof on the surface of the solid support and an amine compound.

8. The solid support of claim 7 wherein the amine compound is selected from morpholine, piperazine, and derivatives thereof.

9. The solid support of claim 8 wherein the amine compound comprises a secondary amine group.

10. The solid support of claim 8 wherein the amine compound comprises a tertiary amine group.

11. The solid support of claim 7 wherein the amine compound is a diamine.

12. The solid support of claim 1 wherein the fluorinated amine compound has the formula:
[NHR¹]ₙ-L
wherein L is a fluorinated hydrocarbyl or ether group comprising up to 8 carbon atoms;
and R¹ is independently hydrogen or a C₁-C₄ alkyl group; and
n is 1 or 2.

13. The solid support of claim 1 wherein the solid support further comprises a second different ligand.

14. The solid support of claim 1 wherein the solid support is particles, magnetic particles, or non-magnetic particles.

15. A solid support comprising a ligand that is the reaction product of an amine compound wherein the amine compound comprises a fluorinated group or a piperazine derivative.

16. (canceled)

17. A kit comprising the solid support of claim 1 and a buffer having a pH of less than 5.5.

18. The kit of claim 17 further comprising a buffer having a pH greater than 6.

19. A method of processing polynucleic acids comprising:
a) providing a solid support comprising ligands with an amide group according to claim 1;
b) exposing the solid support to polynucleic acid molecules in a buffer having a pH less than 5.5 to bind at least a portion of the polynucleic acid molecules to the ligands;
c) exposing the solid support with bound polynucleic acid molecules to a buffer having a pH greater than 6 to release a portion of the bound polynucleic acid molecules from the ligands of the solid support and optionally retaining a portion of the polynucleic acid molecules bound to the solid support;
d) optionally washing the solid support comprising the bound and/or retained bound polynucleic acid molecules;
e) optionally preparing a suspension from the solid support comprising the retained bound polynucleic acid molecules;
f) utilizing the released portion of bound polynucleic acid molecules of c), and/or the retained portion of the polynucleic acid molecules bound to the solid support or the suspension thereof.

20. The method of claim 19 further comprising washing the solid support with a buffer having a pH less than 5.5 after step b).

21. The method of claim 20 wherein f) comprises eluting the released portion of bound polynucleic acids.

22. The method of claim 19 wherein utilizing comprises size separation, purification, quantification, amplification, and preparation of libraries.

23. The method of claim 19 wherein f) comprises preparing a library for nucleic acid sequencing from the eluate.

24. The method of claim 23 wherein f) comprises reacting the polynucleic acid molecules of the eluate with an enzyme.

25. The method of claim 24 wherein the enzyme is bound to a bead.

26. The method of claim 23 wherein the eluate (e.g. of shotgun metagenomic libraries loaded onto a MISeq for sequencing) exhibited enrichment for fragments ranging in size from 100 base pairs to 7000 base pairs.
